(19) United States
(12) Patent Application Publication
(10) Pub. No.: US 2023/0000815 A1
(43) Pub. Date: Jan. 5, 2023

(54) Acid-Labile Chemotherapeutic Compounds and Compositions

(71) Applicant: Veiled Therapeutics LLC, Etta, MS (US)

(72) Inventors: James D. McChesney, Etta, MS (US); Amar G. Chittiboyina, Oxford, MS (US); Suresh Annam, Oxford, MS (US); Saqlain Haider, Oxford, MS (US)

(73) Assignee: Veiled Therapeutics LLC, Etta, MS (US)

(21) Appl. No.: 17/840,455

(22) Filed: Jun. 14, 2022

Related U.S. Application Data
(60) Provisional application No. 63/211,253, filed on Jun. 16, 2021.

(51) Int. Cl. A61K 31/337; A61K 47/54; A61K 9/16; A61P 35/00

(52) U.S. Cl. CPC A61K 31/337; A61K 47/545; A61K 47/543; A61K 9/1617; A61P 35/00

(57) ABSTRACT

The present application discloses an acid labile lipophilic molecular conjugate of cancer chemotherapeutic agents and methods for reducing or substantially eliminating the side effects of chemotherapy associated with the administration of a cancer chemotherapeutic agent to a patient in need thereof.

### CROSS-REFERENCE TO RELATED APPLICATION

This application claims the benefit under 35 USC 119(e) of Application No. 63/211,253, filed Jun. 16, 2021, the entire content of which is incorporated into this application by reference.

### FIELD OF THE INVENTION

The present invention generally relates to compounds and methods for use in treating patients. More particularly, the present invention is directed to molecular conjugates for use in cancer treatment, particularly acid-labile, lipophilic conjugates, methods and intermediates useful in the formation thereof, and methods for treating patients.

### BACKGROUND OF THE INVENTION

A number of anti-cancer drug are currently in the market for the treatment of various cancers. For example, paclitaxel and docetaxel are two promising anti-cancer drugs used to treat breast and ovarian cancers, and which hold promise for the treatment of various other cancers such as skin, lung, head and neck carcinomas. Other promising chemotherapeutic agents are being developed or tested for treatment of these and other cancers. Compounds such as paclitaxel, docetaxel and other taxanes, are of considerable interest. Of special interest are natural product drugs and their synthetic analogs with demonstrated anticancer activity in vitro and in vivo.

However, many identified anti-cancer compounds present a number of difficulties with their use in chemotherapeutic regimens, including the difficulty in targeting cancer tissues, without adversely affecting normal, healthy tissues. For example, paclitaxel exerts its antitumor activity by interrupting mitosis and the cell division process, which occurs more frequently in cancer cells than in normal cells. Nonetheless, a patient undergoing chemotherapy treatment may experience various adverse effects associated with the interruption of mitosis in normal, healthy cells.

Targeted cancer therapies that can selectively kill cancer cells without harming other cells in the body would represent a major improvement in the clinical treatment of cancer. Targeting drugs by conjugation to antibodies for selective delivery to cancer cells has had limited success due to the large size of antibodies (MW=125-150 kilodaltons) and thus their relative inability to penetrate solid tumors.

Accordingly, it would be highly desirable to develop novel compounds and methods for use in directly targeting cancer cells with chemotherapeutic agents in cancer treatment regimens. This could lead to reduction or elimination of toxic side effects, more efficient delivery of the drug to the targeted site, and reduction in dosage of the administered drug and a resulting decrease in toxicity to healthy cells and in the cost of the chemotherapeutic regimen.

One particular approach is the use of anticancer drugs conjugated to tumor molecules. For example, U.S. Pat. No. 6,191,290 to Safavy discloses the use of a taxane moiety conjugated to a receptor ligand peptide capable of binding to tumor cell surface receptors. Safavy indicates that such receptor ligand peptides might be a bombesin/gastrin-releasing peptide (BBN/GRP) receptor-recognizing peptide (BBN [7-13]), a somatostatin receptor-recognizing peptide, an epidermal growth factor receptor-recognizing peptide, a monoclonal antibody or a receptor-recognizing carbohydrate.

These drug molecular conjugates connect these two units with a linker or linkers that provide conjugates with desired characteristics and biological activity, in particular, a conjugate that is stable in systemic circulation but releases cytotoxic agent once internalized into cancer cells or concentrated in the locally acidic tumor environment, which would be expected to exhibit lower toxicity to normal tissues. The resulting conjugate should also be sufficiently stable until it reaches the target tissue, maximizing the targeting effect with reduced toxicity to normal, healthy tissue.

The blood-brain barrier (BBB) is a specialized physical and enzymatic barrier that segregates the brain from systemic circulation. The physical portion of the BBB is composed of endothelial cells arranged in a complex system of tight junctions which inhibit any significant paracellular transport. The BBB functions as a diffusion restraint selectively discriminating against substance transcytosis based on lipid solubility, molecular size and charge thus posing a problem for drug delivery to the brain. Drug delivery across the BBB is further problematic due to the presence of a high concentration of drug efflux transporters (e.g., P-glycoprotein, multi-drug resistant protein, breast cancer resistant protein). These transporters actively remove drug molecules from the endothelial cytoplasm before they even cross into the brain. The methods that are currently employed for drug delivery in treatment of brain malignancies are generally nonspecific and inefficient.

Increased cell proliferation and growth is a trademark of cancer. The increase in cellular proliferation is associated with high turnover of cell cholesterol. Cells requiring cholesterol for membrane synthesis and growth may acquire cholesterol by receptor mediated endocytosis of plasma low density lipoproteins (LDL), the major transporter of cholesterol in the blood, or by de novo synthesis. LDL is taken up into cells by a receptor known as the LDL receptor (LDLR); the LDL along with the receptor is endocytosed and transported into the cells in endosomes. The endosomes become acidified and this releases the LDL receptor from the LDL; the LDL receptor recycles to the surface where it can participate in additional uptake of LDL particles. Evidence suggests that tumors in a variety of tissues have a high requirement for LDL to the extent that plasma LDLs are depleted. The increased import of LDL into cancerous cells may be due to elevated LDL receptors (LDLR) in these tumors. Some tumors known to express high numbers of LDLRs include some forms of leukemia, lung tumors, colorectal tumors and ovarian cancer.

Comparative studies of normal and malignant brain tissues have shown a high propensity of LDLRs to be associated with malignant and/or rapidly growing brain cells and tissues. Some studies suggest that rapidly growing brain cells such as those seen in early development and in aggressively growing brain tumors exhibit increased expression of LDLRs due to their increased requirement for cholesterol.

Among the problematic and inefficiently treated brain cancers is glioblastoma multiforme (GBM). This devastating brain tumor is 100% fatal. Moreover, over 85% of total primary brain cancer-related deaths are due to GBM. Current therapies rely on a multimodal approach including neurosurgery, radiation therapy and chemotherapy. Even the best efforts using these approaches have resulted in only a modest increase in survival time for patients afflicted with this tumor. GBM cells in culture have high numbers of low density lipoprotein receptors (LDLR). Since this receptor is nearly absent in neuronal cells and normal glial cells, it represents an ideal target for the delivery of therapeutic agents such as cytotoxins or radiopharmaceuticals. Efforts to improve existing therapies or to develop new ones have not been successful and the outcome of treatment for malignant gliomas is only modest, with a median survival time of approximately 10 months.

Unlike normal brain cells that have few LDL receptors, GBM cells in culture have high numbers of LDL receptors on their surface. Other cancers are likely to also have high expression of LDLR due to the highly proliferative nature of the cancerous tissue and need for cholesterol turnover. This suggests that the LDL receptor is a potential unique molecular target in GBM and other malignancies for the delivery of anti-tumor drugs via LDL particles.

Maranhao and coworkers have demonstrated that a cholesterol-rich microemulsion or nanoparticle preparation termed LDE concentrates in cancer tissues after injection into the bloodstream. R. C. Maranhao et al. Improvement of paclitaxel therapeutic index by derivatization and association to a cholesterol-rich microemulsion: in vitro and in vivo studies. Cancer Chemotherapy and Pharmacology 55: 565-576 (2005). The cytotoxicity, pharmacokinetics, toxicity to animals and therapeutic action of a paclitaxel lipophilic derivative associated to LDE were compared with those of commercial paclitaxel. Results showed that LDE-paclitaxel oleate was stable. Maranhao and coworkers showed LDE-paclitaxel oleate is a stable complex and compared with paclitaxel, toxicity is considerably reduced and activity is enhanced which may lead to improved therapeutic index in clinical use.

Capturing the potential of selective and specific delivery of chemotherapeutic compounds to cancer tissues via their over expression of LDL receptors and consequent high uptake of LDL particles from the systemic circulation, requires that the cancer chemotherapeutic agent have high lipophilicity so as to remain entrapped in the lipid core of the LDL particle and not diffuse into the plasma to lead to toxic side effects from exposure of normal tissues to the agent. Once the LDL particle with its chemotherapeutic payload has entered the cancer cell via LDL receptor mediated uptake and enters into the acidic environment of the endosome/lysosome cascade, the LDL receptor is disassociated from the LDL particle and is recycled to the cell surface and the LDL particle releases its lipid contents and its lipophilic chemotherapeutic agent to the enzymes and acidic environment of the endosome become lysosome. Few cancer chemotherapeutic agents are intrinsically sufficiently lipophilic to be retained adequately within the lipid core of the LDL particle. This creates a need for suitable lipophilic derivatives of the cancer chemotherapeutic agent which have high stability in normal systemic circulation and retention in the lipid core of the LDL particles but readily release the active chemotherapeutic agent in the acidic environment of the endosome/lysosome. The compounds of the present invention address this need.

### Definitions

The term “diastereoisomer” refers to any group of four or more isomers occurring in compounds containing two or more asymmetric carbon atoms. Compounds that are stereoisomers of one another, but are not enantiomers are called diastereosiomers.

The clause “diastereoisomerically pure” refers to a compound or a diastereoisomer having a single diastereoisomer that is at least about 95%, 96%, 97%, 98%, 99%, 99.5%, 99.7%, 99.8% or at least 99.9% pure or free from the presence of any of the other possible diastereoisomers of the compound.

“Pharmaceutically acceptable excipient” or “pharmaceutically acceptable salts” as used herein, means the excipient or salts of the compounds disclosed herein, that are pharmaceutically acceptable and provides the desired pharmacological activity. These excipients and salts include acid addition salts formed with inorganic acids such as hydrochloric acid, hydrobromic acid, phosphoric acid, and the like. The salt may also be formed with organic acids such as acetic acid, propionic acid, hexanoic acid, glycolic acid, lactic acid, succinic acid, malic acid, citric acid, benzoic acid and the like.

“Therapeutically effective amount” means a drug amount that elicits any of the biological effects listed in the specification.

### SUMMARY OF THE INVENTION

In one embodiment, there is provided new and useful compositions of molecular conjugates of hydroxyl-bearing cancer chemotherapeutic agents (HBCCA). In another embodiment, there is provided compositions of acid labile, lipophilic molecular conjugates of cancer chemotherapeutic agents for use in treating cancer. In another embodiment, there is provided intermediate compounds for use in forming molecular conjugates, such as acid labile, lipophilic pro-drug conjugates, for use in treating cancer. In another embodiment, there is provided efficient methods for the preparation of acid labile, lipophilic drug conjugates. In another embodiment, there is provided methods for administering chemotherapeutic agents to patients that reduce or substantially eliminate side effects conventionally experienced by cancer patients. In another embodiment, there is provided methods for concentrating chemotherapeutic agents in cancer cells of a patient.

In one embodiment of the present application, there is provided an acid labile lipophilic molecular conjugate (ALLMC) of the formulae:

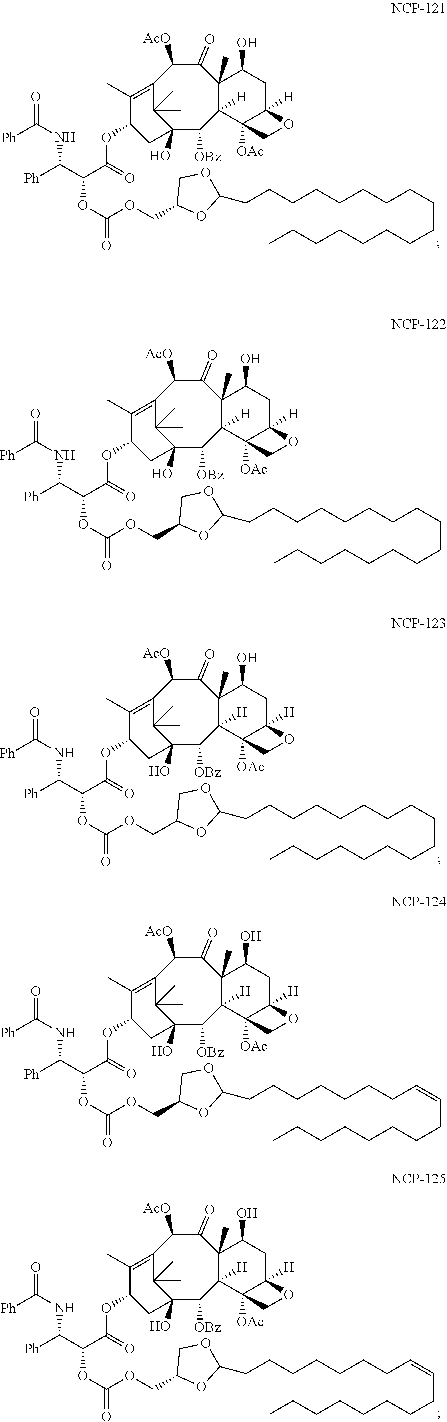

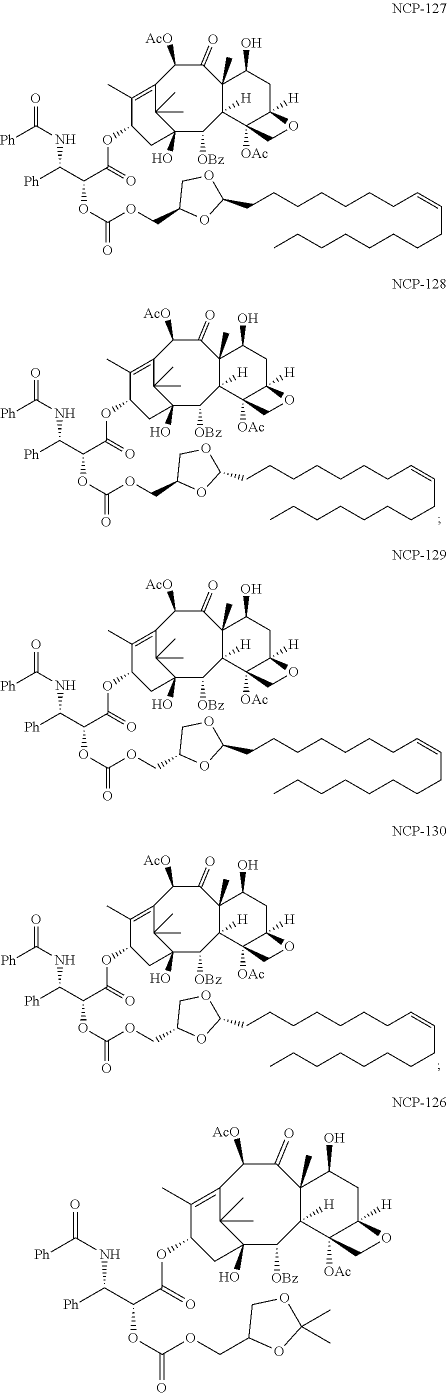

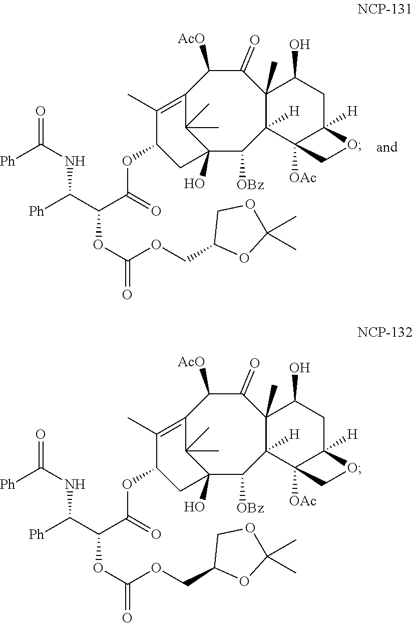

and their isolated diastereoisomers or mixtures thereof; or a pharmaceutically acceptable salt thereof.

In one aspect, the acid labile lipophilic molecular conjugate is (2aR,4S ,4aS ,6R,9S,11S,12S,12aR,12bS)-9-(((2R,3S)-3-benzamido-2-((((2,2-dimethyl-1,3-dioxolan-4-yl)methoxy)carbonyl)oxy)-3 -phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-126). In another aspect, the acid labile lipophilic molecular conjugate is (2aR,4S,4aS,6R,9S,11S,12S,12aR,12bS)-9-(((2R,3S)-3-benzamido-2-(((((S)-2,2-dimethyl-1,3-dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-131). In another aspect, the acid labile lipophilic molecular conjugate is (2aR,4S,4aS,6R,9S,11S,12S,12aR,12bS)-9-(((2R,3S)-3-benzamido-2-(((((R)-2,2-dimethyl-1,3-dioxolan-4- yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo [1,2-b]oxete-6,12b(2aH)-diyl diacetate (NCP-132). In another aspect of the acid labile lipophilic molecular conjugate, the hydroxyl bearing cancer chemotherapeutic agent is paclitaxel or cabazitaxel. As used herein, the “hydroxyl bearing cancer chemotherapeutic agent” is paclitaxel or cabazitaxel having the 2′-hydroxyl group that is conjugated to the carbonate group (—OC(O)O—). In another embodiment, there is provided a pharmaceutical composition comprising: a) a therapeutically effective amount of any of the above compounds in the form of a single diastereoisomer; and b) a pharmaceutically acceptable excipient.

In another embodiment, there is provided a method for the treatment of cancer in a patient comprising administering to the patient a therapeutically effective amount of any of the above cited compound or composition, to a patient in need of such treatment. In another aspect of the method, the cancer is selected from the group consisting of leukemia, neuroblastoma, glioblastoma, cervical, colorectal, pancreatic, renal and melanoma. In another aspect, the cancer is selected from the group consisting of lung, breast, prostate, ovarian and head and neck. In another aspect, the method provides at least a 10% to 50% diminished degree of resistant expressed by the cancer cells when compared with the non-conjugated hydroxyl bearing cancer chemotherapeutic agent that is paclitaxel or cabazitaxel.

In another embodiment, there is provided a method for reducing or substantially eliminating the side effects of chemotherapy associated with the administration of paclitaxel or cabazitaxel to a patient, the method comprising administering to the patient a therapeutically effective amount of an acid labile lipophilic molecular conjugate (ALLMC) of any one of the above cited compounds. In another aspect, the method provides a higher concentration of the paclitaxel or cabazitaxel in a cancer cell of the patient. In another aspect, the method delivers a higher concentration of paclitaxel or cabazitaxel in the cancer cell, when compared to the administration of a non-conjugated cancer chemotherapeutic agent that is paclitaxel or cabazitaxel to the patient, by at least 5%, 10%, 20% or at least 50%.

In another embodiment, there is provided a stable, synthetic low density lipoprotein (LDL) solid nanoparticle comprising: a) an acid labile lipophilic molecular conjugate (ALLMC) of the formulae NCP-121, NCP-122, NCP-123, NCP-124, NCP-125, NCP-127, NCP-128, NCP-129, NCP-130, NCP-126 and NCP-131 and NCP-132 and their isolated diastereoisomers or mixtures thereof; or a pharmaceutically acceptable salt thereof; b) phospholipids (PL) wherein the phospholipids is selected from the group consisting of phosphotidylcholine, phosphotidylethanolamine, symmetric or asymmetric 1,2-diacyl-sn-glycero-3-phosphorylcholines, 1,2-dimyristoyl-sn-glycero-3-phosphorylcholine, 1,2-dimyristoyl-sn-glycero-3-phosphorylethanolamine, egg phospholipids, egg phosphatidyl glycerol, dipalmitoylphosphatidyl glycerol, egg lecithin, soy lecithin, lecithin (NOS) and mixtures thereof; and c) a triglyceride (TG) selected from the group consisting of MIGLYOL 812 N, triacetin, tripropionin, tributyrin, triisovalerin, triisovalerin, tricapronin, triheptylin, tricaprylin, trinonylin, tricaprinin, and triundecylin; wherein the LDL solid nanoparticle has a mean particle size of 40-80 nm. In one variation, the triglyceride used may be Miglyol 812N (a C8/C10 triglyceride (MCT oil)), or may be another triglyceride esters or other medium-chain triglycerides as disclosed herein.

In another aspect of the above stable, synthetic low density lipoprotein (LDL) solid nanoparticle, the acid labile lipophilic molecular conjugate is (2aR,4S ,4aS ,6R,9S,11S,12S,12aR,12bS)-9-(((2R,3S)-3-benzamido-2-((((2,2-dimethyl-1,3-dioxolan-4-yl)methoxy)carbonyl)oxy)-3 -phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-126). In another aspect of the above stable, synthetic low density lipoprotein (LDL) solid nanoparticle, the acid labile lipophilic molecular conjugate is (2aR,4S,4aS,6R,9S,11S,12S,12aR,12bS)-9-(((2R,3S)-3-benzamido-2-(((((S)-2,2-dimethyl-1,3-dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyl diacetate (NCP-131). In another aspect of the above stable, synthetic low density lipoprotein (LDL) solid nanoparticle, the acid labile lipophilic molecular conjugate is (2aR,4S ,4aS ,6R,9S,11S,12S,12aR,12bS)-9-(((2R,3S)-3-benzamido-2-(((((R)-2,2-dimethyl-1,3-dioxolan-4-yl)methoxy)carbonyl)oxy)- 3 -phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-132). In one aspect of the above stable, synthetic low density lipoprotein (LDL) solid nanoparticle, the nanoparticle has a mean size distribution of 60 nm.

In another embodiment, there is provided a pharmaceutical composition comprising: a) a therapeutically effective amount of a compound of the above, in the form of a single diastereoisomer; and b) a pharmaceutically acceptable excipient. In another aspect, the pharmaceutical composition is adapted for oral administration; or as a liquid formulation adapted for parenteral administration. In another aspect, the composition is adapted for administration by a route selected from the group consisting of orally, parenterally, intraperitoneally, intravenously, intraarteriall, transdermally, intramuscularly, rectally, intranasally, liposomally, subcutaneously and intrathecally. In another embodiment, there is provided a method for the treatment of cancer in a patient comprising administering to the patient a therapeutically effective amount of a compound or composition of any of the above compound or composition, to a patient in need of such treatment. In one aspect of the method, the cancer is selected from the group consisting of leukemia, neuroblastoma, glioblastoma, cervical, colorectal, pancreatic, renal and melanoma. In another aspect of the method, the cancer is selected from the group consisting of lung, breast, prostate, ovarian and head and neck. In another aspect of the method, the method provides at least a 10%, 20%, 30%, 40%, or at least a 50% diminished degree of resistance expressed by the cancer cells when compared with the non-conjugated hydroxyl bearing cancer chemotherapeutic agent.

In another embodiment, there is provided a method for reducing or substantially eliminating the side effects of chemotherapy associated with the administration of a cancer chemotherapeutic agent to a patient, the method comprising administering to the patient a therapeutically effective amount of an acid labile lipophilic molecular conjugate of the formulae as disclosed herein.

In one aspect, the method provides a higher concentration of the cancer chemotherapeutic agent in a cancer cell of the patient. In another aspect, the method delivers a higher concentration of the cancer chemotherapeutic agent in the cancer cell, when compared to the administration of a non-conjugated cancer chemotherapeutic agent to the patient, by at least 5%, 10%, 20%, 30%, 40% or at least 50%.

In another embodiment, there is provided a method for concentrating a cancer chemotherapeutic agent in selected target cells of a patient using the acid labile, lipophilic molecular conjugates of the present application in a nanoparticulate lipid emulsion resembling a LDL particles or “pseudo-LDL particles”. In another embodiment, the method comprises administering to a patient a selected dose of a therapeutically effective amount of the acid labile, lipophilic molecular conjugate of a cancer chemotherapeutic agent dissolved in the lipid core of the pseudo-LDL particles.

Also included in the above embodiments, aspects and variations are salts of amino acids such as arginate and the like, gluconate, and galacturonate. Certain of the compounds of the present invention can exist in unsolvated forms as well as solvated forms, including hydrated forms, and are intended to be within the scope of the present invention. Also provided are pharmaceutical compositions comprising pharmaceutically acceptable excipients and a therapeutically effective amount of at least one compound of this invention.

Pharmaceutical compositions of the compounds of this invention, or derivatives thereof, may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, 5% dextrose in water or buffered sodium or ammonium acetate solution. Such formulations are especially suitable for parenteral administration but may also be used for oral administration. Excipients, such as polyvinylpyrrolidinone, gelatin, hydroxycellulose, acacia, polyethylene glycol, mannitol, sodium chloride, or sodium citrate, may also be added. Alternatively, these compounds may be encapsulated, tableted, or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols or water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulation, and compressing, when necessary, for tablet forms; or milling, mixing, and filling for hard gelatin capsule forms. When a liquid carrier is used, the preparation will be in the form of a syrup, elixir, emulsion, or an aqueous or non-aqueous suspension. Such a liquid formulation may be administered directly p.o. or filled into a soft gelatin capsule. Suitable formulations for each of these methods of administration may be found in, for example, Remington: The Science and Practice of Pharmacy, A. Gennaro, ed., 20th edition, Lippincott, Williams & Wilkins, Philadelphia, Pa.

These and other objects of the present invention will become more readily appreciated and understood from a consideration of the following detailed description of the exemplary embodiments of the present invention when taken together with the accompanying drawings and figures. The entire disclosures of all documents cited throughout this application are incorporated herein by reference.

### DETAILED DESCRIPTION OF THE EXEMPLARY EMBODIMENTS

The following procedures may be employed for the preparation of the compounds of the present invention. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

In some cases, protective groups may be introduced and finally removed. Suitable protective groups for amino, hydroxy, and carboxy groups are described in Greene et al., Protective Groups in Organic Synthesis, Second Edition, John Wiley and Sons, New York, 1991. Standard organic chemical reactions can be achieved by using a number of different reagents, for examples, as described in Larock: Comprehensive Organic Transformations, VCH Publishers, New York, 1989.

### EXPERIMENTAL

### General Procedures

The chemicals and reagents were purchased from Sigma Aldrich (MO, USA). Paclitaxel (>99%) was purchased from LC Laboratories (Woburn, Mass,). All the IR spectra were recorded on an Agilent 630 FTIR (Agilent Technologies, CA, USA). ¹H and ¹³C NMR spectra were recorded on Bruker (400 and 500 MHz; Bruker Biospin, MA, USA) spectrometers, and chemical shifts were expressed as ppm against tetramethylsilane as an internal reference. Mass spectra were recorded on Agilent 1290 UHPLC and 6120 MS (Agilent Technologies), and column chromatography was performed on (Merck, MA, USA) silica gel. All reactions were carried out under an argon atmosphere unless otherwise stated. Thin-layer chromatography (TLC) was performed on precoated silica gel G and GP Uniplates. The plates were visualized with a 254-nm UV light, iodine chamber, or charring with Phosphomolybdic acid (PMA). General Procedure for Synthesis of Acid Labile, Lipophilic Molecular Conjugates of Cancer Chemotherapeutic Agents.

### Formation of Acid-Labile Lipophilic Conjugates

Acid labile lipophilic conjugates were characterized by a combination of HPLC and High Resolution Mass Spectrometry. Specifics are provided with each compound.

ART 207 was prepared by following the procedure as outlined in Method A. HPLC retention time 6.06, Method; Taxane conjugates_MKG17 (Synergy column, ACN/H₂O 60/40 to 100% ACN 10 min, 2 min 100% ACN, 230 nm, 1.5 ml/min, 30° C., 15 min). +TOF MS: m/z 1220.6156 [M+] and m/z 1237.6382 [M+18] (M+NH4⁺).

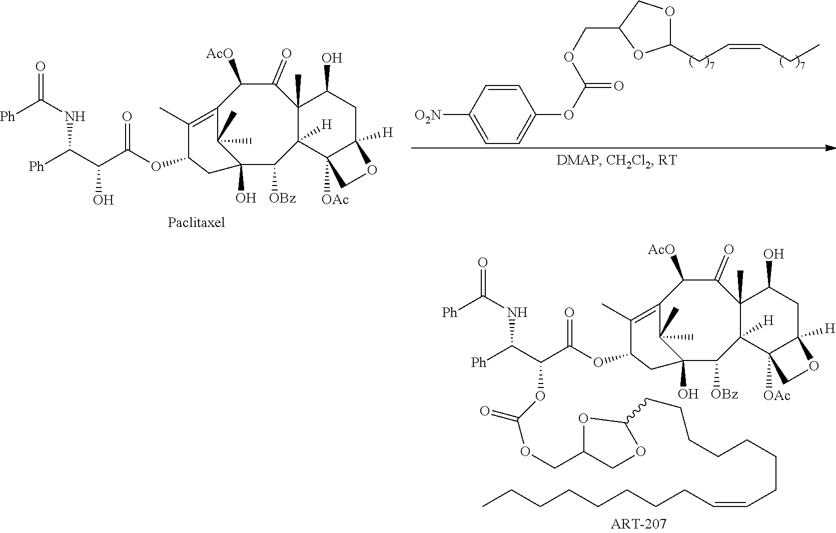

HPLC analysis of the compounds shows a sharp, single peak using various optimized HPLC conditions, including:
1) C18 column, ACN/H₂O 50/50 to 100% ACN 10 min, 2 min 100% ACNH, 230 nm, 1.5 ml/min, 30° C., 16 min;
2) Synergy column, MeOH/H₂O 75/25 to 100% MeOH 10 min, 2 min 100% MeOH, 230 nm, 1.5 ml/min, 30° C., 15 min;
3) Synergy column, ACN/H₂O 50/50 3 min, 80-100% ACN/H₂O 10 min, 2 min 100% ACN, 230 nm, 1.5 ml/min, 30° C., 15 min;
4) Synergy column, 70-100% ACN/H₂O 10 min, 100% ACN 2 min, 230 nm, 1.5 ml/min, 30° C., 15 min;
5) C18 column, MeOH/H₂O 95/5 to 100% MeOH 10 min, 2 min 100% MeOH, 230 nm, 1.5 ml/min, 30° C., 16 min; and
6) Synergy column, ACN/H₂O 80/20 10 min, 100% ACN 2 min, 230 nm, 1.5 ml/min, 30° C., 15 min.

The compounds of the present application, including the lipophilic prodrugs (ART-207 & NCP-121 to NCP-132) were designed and prepared to compare their biosimilarities with protein-bound paclitaxel, Abraxane®. Commercially available methyl oleate 1 was intentionally over-reduced to the corresponding oleyl alcohol 2 using DIBAL (−78° C.), and the resulting carbinol was oxidized with pyridinium chlorochromate at 50° C. to olealdehyde 3 as a colorless oil in 76%. The linker, racemic solketal carbonate (±)-5, was prepared in 70% yield by reacting racemic solketal (4) with 4-nitrophenyl chloroformate in the presence of pyridine and DMAP as outlined in Scheme 1. The enantiomerically pure solketal carbonates (−)-5 and (+)-5 were also synthesized using identical chemical transformations starting from the corresponding enantiomerically pure R(−)-solketal [(−)-4] and S(+)-solketal [(+)-4], respectively (Scheme 1). The Amberlyst-15 catalyzed transacetalization between olealdehyde 3 and solketal carbonate 5 resulted in the formation of the 1,3-dioxolane intermediates 6 with a 2:1 preference to an anti-isomer. Prior acetalization followed by carbonylation with 4-nitrophenyl chloroformate was not effective, as initial transacetalization resulted in inseparable anti- and syn -isomers of both 5-membered and 6-membered 1,3-dioxolanes in 5.6:2.9:1.1:1 ratios. The saturated congener, stearaldehyde, prepared from methyl stearate, was also converted to the corresponding racemic and diastereomerically pure carbonates 8 in 65% overall yield by the following carbonylation and transacetalization process.

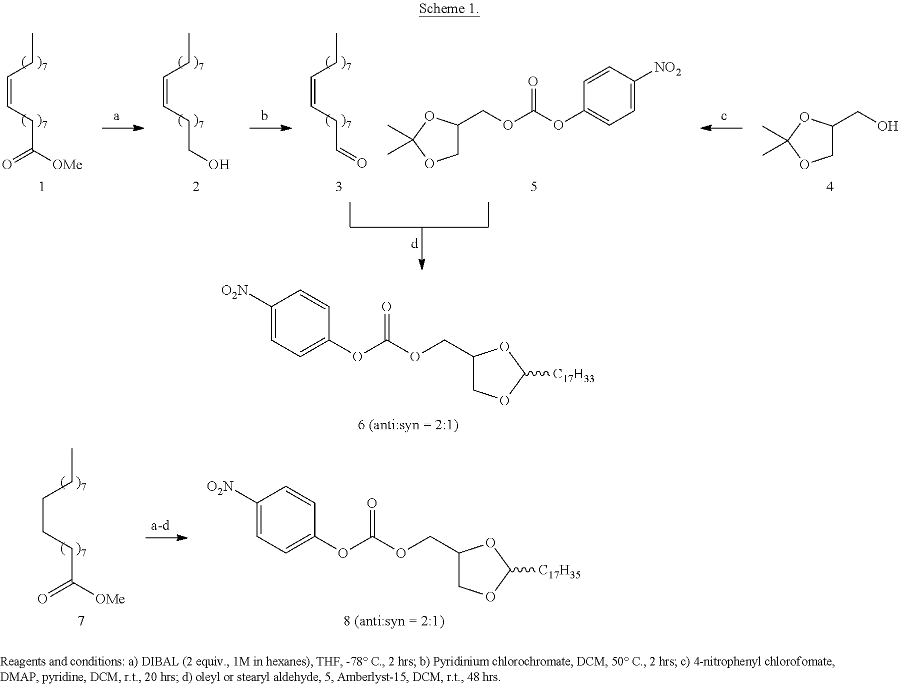

In a separate attempt to resolve anti- and syn-isomers of 1,3-dioxolane carbonates, (−)-6 and (+)-6, further into two sets of enantiomerically pure, single isomers, a recycling preparative high-pressure liquid chromatography was applied using Phenomenex Prep HPLC column. The mobile phase consisting with hexanes and THF in 94:6 (v/v) was found to be an ideal isocratic solvent system to produce enantiomerically pure single isomers [(−)-6 to (−)-6a and (−)-6s; (+)-6 to (+)-6a and (+)-6s; a stands for anti-isomer, and s stands for syn-isomer] after four rounds of recycling with the eluent. The purity of these single enantiomers was established with full characterization and chromatographic spectral data. A total of 10 divergent, readily reactive lipid-attached solketal nitrophenyl carbonates (5, 6, and 8) suitable for conjugation with the cytotoxics were prepared in gram-scale quantities.

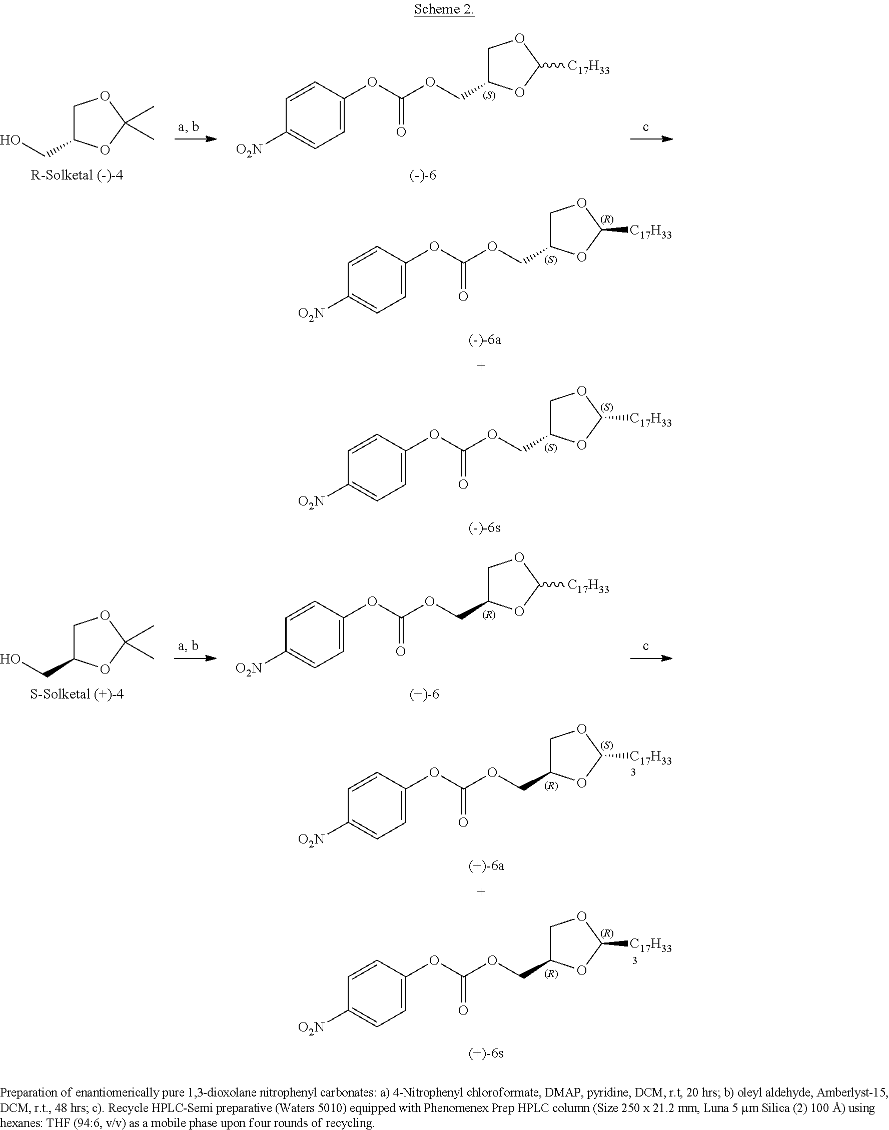

Selective conjugation at C2′-position of paclitaxel with 1,3-dioxolane nitrophenyl carbonates (5, 6 and 8) was achieved in almost quantitative yields with DMAP as a base. By switching the appropriate lipid carbonate linker, many conjugates, including enantiomerically enriched, were synthesized as delineated in the Table-1. For example, DMAP-mediated reaction between equimolar amount of paclitaxel and 1,3-dioxolane intermediates (±)-6 resulted in 86% of ART-207 as a white solid. Analogs NCP-124 and NCP-125 were synthesized from the intermediates (−)-6 and (+)-6 respectively. All other paclitaxel conjugates were prepared by simply changing the 1,3-dioxolane nitrophenyl carbonate linker to achieve 13 unique, lipophilic, acid-labile prodrugs.

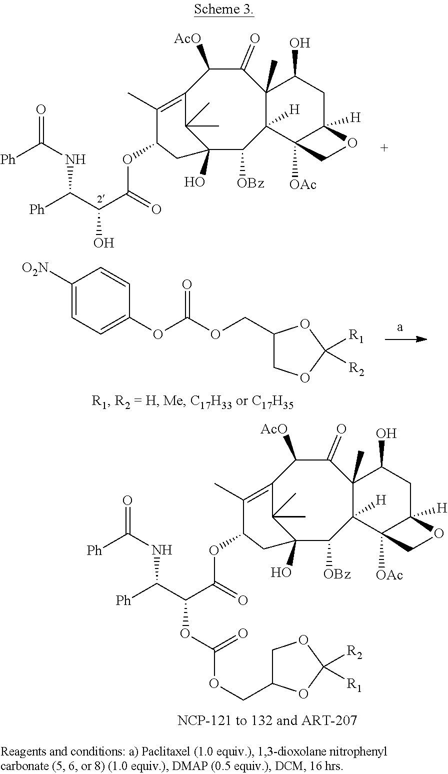

TABLE 1
Synthesis of various isomerically pure late-
stage divergent conjugates of paclitaxel.

| | 1,3- | | | | |
|---|---|---|---|---|---|
| S | dioxolane | | | Yield | |
| No | carbonate* | R₁ | R₂ | (%) | Compound |
| 1 | (−)-8 | H | C₁₇H₃₅ | 87 | NCP-121 |
| 2 | (+)-8 | H | C₁₇H₃₅ | 84 | NCP-122 |
| 3 | (±)-8 | H | C₁₇H₃₅ | 86 | NCP-123 |
| 4 | (±)-6 | H | C₁₇H₃₃ | 86 | ART-207 |
| 5 | (−)-6 | H | C₁₇H₃₃ | 75 | NCP-124 |
| 6 | (+)-6 | H | C₁₇H₃₃ | 76 | NCP-125 |
| 7 | (−)-6s | H | C₁₇H₃₃ | 95 | NCP-127 |
| 8 | (−)-6a | H | C₁₇H₃₃ | 92 | NCP-128 |
| 9 | (+)-6a | H | C₁₇H₃₃ | 90 | NCP-129 |
| 10 | (+)-6s | H | C₁₇H₃₃ | 92 | NCP-130 |
| 11 | (±)-5 | Me | Me | 80 | NCP-126 |
| 12 | (+)-5 | Me | Me | 78 | NCP-131 |
| 13 | (−)-5 | Me | Me | 82 | NCP-132 |

*1,3-dioxolane carbonates were tabulated based on the targeted compounds.

(2aR,4S,4aS,6R,9S,11S,12S,12aR,12bS)-9-(((2R,3S)-3-benzamido-2-(((((4R)-2-heptadecyl-1,3-dioxolan-4- yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyl diacetate (NCP-121).

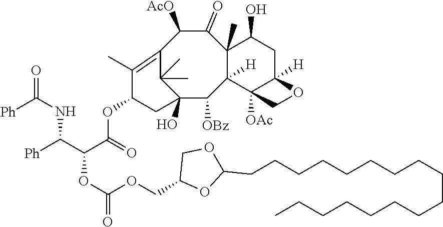

IR: (cm⁻¹; neat): 2927, 2853, 1718, 1654, 1235. [α]_{D}²⁰=−46.6; (c2, CHCl₃). ¹H NMR (400 MHz, CDCl₃, mixture of two isomers*) δ 8.15-8.13; (m, 2H), 7.75-7.73; (m, 2H), 7.63-7.59; (m, 1H), 7.53-7.49; (m, 3H), 7.45-7.34; (m, 7H), 6.92; (dd, J=9.4, 2.4 Hz, 1H), 6.29; (s, 2H), 6.00; (dd, J=9.4, 2.5 Hz, 1H), 5.69; (d, J=7.0 Hz, 1H), 5.43-5.42; (m, 1H), 4.99-4.96; (m, 2H), 4.88; (t, J=6.6, 1H), 4.47 — 4.42; (m, 1H) 4.34-4.30; (m, 1H), 4.29-4.23; (m, 1H), 4.23-4.17; (m, 2H), 4.16-4.08; (m, 2H), 3.92-3.88; (m, 0.60H), 3.82; (d, J=7.4 Hz, 1H), 3.59-3.55; (m, 0.50H), 2.60-2.53; (m, 1H), 2.50; (d, J=4.1 Hz, 1H), 2.46; (s, 3H), 2.43-2.37; (m, 1H), 2.23; (s, 3H), 2.04; (s, 1H), 1.95-1.92; (m, 3H), 1.90-1.85; (m, 1H), 1.83; (s, 1H), 1.72; (s, 2H), 1.68; (s, 3H), 1.65-1.62; (m, 1H), 1.42-1.38; (m, 2H), 1.27-1.25; m, 29H), 1.14; (s, 3H), 0.88; (t, J=6.7 Hz, 3H). ¹³C NMR (101 MHz, CDCl₃) δ 203.77, 171.22, 171.18, 169.85, 167.77, 167.16, 167.00, 154.12, 154.04, 142.56, 136.66, 133.66, 133.49, 132.91, 132.06, 130.24, 129.20, 129.14, 128.75, 128.71, 128.55, 127.17, 126.56, 105.72, 105.03, 84.45, 81.09, 79.12, 75.59, 75.12, 73.02, 72.96, 72.14, 69.12, 68.45, 66.92, 66.78, 60.41, 58.52, 52.65, 45.60, 43.21, 35.58, 33.87, 33.84, 31.93, 29.71, 29.66, 29.57, 29.54, 29.51, 29.37, 26.84, 23.95, 23.87, 22.73, 22.70, 22.17, 21.05, 20.83, 14.81, 14.21, 14.14, 9.62. HRMS (ESI): m/z (M+Na)⁺ calcd. for C₆₉H₉₁NO₁₈Na, 1244.6133; found, 1244.6120.

(2aR,4S,4aS,6R,9S,11S,12S,12aR,12bS)-9-(((2R,3S)-3-benzamido-2-(((((4S)-2-heptadecyl-1,3-dioxolan-4- yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyl diacetate (NCP-122).

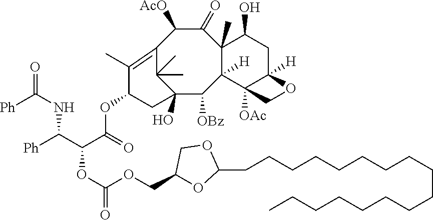

IR: (cm⁻¹; neat): 2924, 2851, 1718, 1664, 1369, 1235. [α]_{D}²⁰=−40.0 (c2, CHCl₃). ¹H NMR (400 MHz, CDCl₃) δ 8.15-8.13; (m, 2H), 7.73-7.75; (m, 2H), 7.60; (t, J=7.4 Hz, 1H), 7.53-7.48; (m, 3H), 7.45-7.34; (m, 7H), 6.95; (dd, J=9.4, 5.0 Hz, 1H), 6.29; (s, 2H), 6.00; (dd, J=9.4, 2.5 Hz, 1H), 5.69; (d, J=7.1 Hz, 1H), 5.42; (d, J=2.6 Hz, 1H), 4.98; (dd, J=9.8, 2.2 Hz, 1H), 4.93; (d, J=4.8 Hz, 0.37H), 4.87; (t, J=4.8 Hz, 0.47H), 4.45-4.41; (m, 1H), 4.32; (d, J=8.6 Hz, 1H), 4.29-4.23; (m, 1H), 4.21; (dd, J=6.4, 2.0 Hz, 2H), 4.16-4.13; (m, 2H), 3.90; (dd, J=8.5, 7.0 Hz, 0.50H), 3.82-3.79; (m, 1H), 3.61; (dd, J=8.5, 6.8 Hz, 0.50H), 2.53; (dd, J=6.2, 3.7 Hz, 2H), 2.46; (d, J=1.8 Hz, 3H), 2.43-2.37; (m, 1H), 2.22; (s, 3H), 2.03; (s, 1H) 1.93; (s, 3H), 1.89; (s, 2H), 1.81; (s, 2H), 1.68; (s, 3H), 1.63-1.60; (m, 1H), 1.43-1.33; (m, 2H), 1.25; (m, 30H), 1.14; (s, 3H), 0.88; (t, J=6.8 Hz, 3H). ¹³C NMR (101 MHz, CDCl₃) δ 203.78, 171.23, 171.19, 169.85, 167.78, 167.74, 167.23, 166.99, 154.09, 154.03, 142.56, 142.53, 136.66, 133.66, 133.47, 132.92, 132.07, 130.23, 129.21, 129.13, 128.74, 128.71, 128.56, 127.18, 126.57, 105.67, 105.09, 84.45, 81.10, 79.10, 75.59, 75.12, 72.88, 72.82, 72.14, 68.96, 68.32, 66.66, 60.41, 58.51, 52.69, 45.59, 43.21, 35.59, 33.88, 33.83, 31.93, 29.70, 29.68, 29.66, 29.57, 29.54, 29.50, 29.36, 26.83, 23.96, 23.84, 22.73, 22.70, 22.17, 21.05, 14.79, 14.20, 9.63. HRMS (ESI): m/z (M+Na)⁺ calcd. for C₆₉H₉₁NO₁₉Na, 1244.6133; found, 1244.6116.

(2aR,4S,4aS,6R,9S,11S,12S,12aR,12bS)-9-(((2R,3S)-3-benzamido-2-((((2-heptadecyl-1,3-dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyldiacetate (NCP-123).

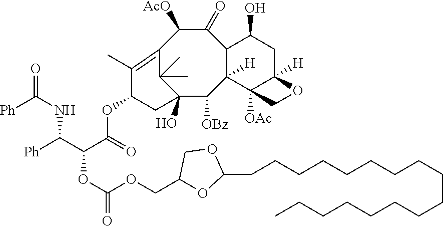

IR: (cm⁻¹; neat): 2920, 2849, 1718, 1239. [α]_{d}²⁰=−44.4; (c2, CHCl₃). ¹H NMR (400 MHz, CDCl₃) δ 8.14; (dd, J=7.4, 1.6 Hz, 2H), 7.75-7.73; (m, 2H), 7.62-7.57; (m, 1H), 7.53-7.48; (m, 3H), 7.45-7.34; (m, 7H), 6.95-6.91; (m, 1H), 6.30; (s, 2H), 6.01-5.98; (m, 1H), 5.69; (d, J=7.1 Hz, 1H), 5.44-5.42; (m, 1H), 4.99-4.94; (m, 1H), 4.89-4.86; (m, 1H), 4.44; (dd, J=10.9, 6.6 Hz, 1H), 4.32; (d, J=8.7 Hz, 1H), 4.30-4.23; (m, 1H), 4.20; (ddd, J=8.8, 4.9, 2.1 Hz, 2H), 4.19-4.15; (m, 1H), 4.12; (dd, J=6.9, 2.2 Hz, 1H), 3.90; (dd, J=8.6, 6.9 Hz, 0.63H), 3.83-3.78; (m, 2H), 3.63-3.55; (m, 0.46H), 2.58-2.50; (m, 1H), 2.46; (s, 3H), 2.44-2.38; (m, 1H), 2.23; (s, 4H), 2.04; (s, 1H), 1.93; (t, J=1.7 Hz, 3H), 1.90-1.85; (m, 1H), 1.68; (s, 3H), 1.66-1.62; (m, 1H), 1.40-1.35; (m, 2H), 1.32-1.22; (m, 32H), 1.14; (s, 3H), 0.88; (t, J=6.8 Hz, 3H). ¹³C NMR (101 MHz, CDCl₃) δ 203.78, 171.24, 169.85, 167.77, 167.17, 167.01, 154.10, 154.04, 142.58, 136.67, 133.67, 133.48, 132.90, 132.07, 130.24, 129.20, 129.14, 128.75, 128.71, 128.56, 127.17, 126.56, 114.98, 105.72, 105.68, 105.10, 105.03, 84.45, 81.10, 79.14, 75.59, 75.12, 73.02, 72.96, 72.88, 72.82, 72.14, 69.12, 68.96, 68.45, 66.92, 66.83, 66.67, 60.41, 58.52, 52.68, 52.64, 45.59, 43.21, 35.60, 35.55, 33.88, 33.84, 31.93, 29.71, 29.66, 29.57, 29.54, 29.51, 29.37, 26.84, 23.96, 23.87, 23.85, 22.73, 22.70, 22.18, 21.05, 14.80, 14.21, 9.62. HRMS (ESI): m/z (M+Na)⁺ calcd. for C₆₉H₉₁NO₁₉Na, 1244.6133; found, 1244.6111.

(2aR,4S,4aS,6R,9S,11S,12S,12aR,12bS)-9-(((2R,3S)-3-benzamido-2-((((2-(Z)-heptadec-8-en-1-yl)-1,3-dioxolan-4- yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyldiacetate (ART-207).

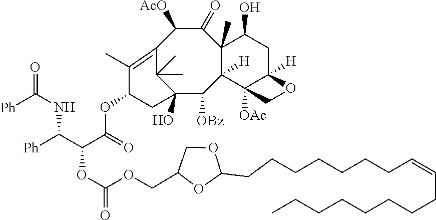

¹H NMR (400 MHz, CDCl₃) δ 8.17-8.11; (m, 2H), 7.74; (dt, J=8.3, 1.2 Hz, 2H), 7.63-7.57; (m, 1H), 7.53-7.49; (m, 3H), 7.45-7.35; (m, 7H), 6.92; (dt, J=8.4, 4.1 Hz, 1H), 6.33-6.24; (m, 2H), 6.00; (dd, J=9.2, 2.4 Hz, 1H), 5.69; (d, J=7.1 Hz, 1H), 5.44-5.42; (m, 1H), 5.38-5.30; (m, 2H), 5.00-4.92; (m, 2H), 4.47-4.44; (m, 1H), 4.35-4.24; (m, 2H), 4.24-4.18; (m, 2H), 4.16-4.09; (m, 1H), 3.85-3.77; (m, 2H), 2.61-2.51; (m, 1H), 2.49; (dd, J=4.1, 1.4 Hz, 1H), 2.46; (dd, J=2.6, 1.3 Hz, 3H), 2.44-2.36; (m, 1H), 2.23; (m, 4H), 2.00; (q, J=6.5 Hz, 4H), 1.93; (q, J=1.4 Hz, 3H), 1.90-1.84; (m, 1H), 1.81; (d, J=1.3 Hz, 1H), 1.71-1.65; (m, 6H), 1.38-1.41; (m, 3H), 1.33-1.23; (m, 22H), 1.14; (s, 3H), 0.89-0.87; (m, 3H). ¹³C NMR (101 MHz, CDCl₃) δ 203.79, 171.27, 169.86, 167.76, 167.14, 167.04, 142.61, 136.66, 133.69, 133.47, 132.89, 132.08, 130.24, 129.99, 129.78, 129.18, 129.15, 128.76, 128.72, 128.56, 127.17, 126.54, 105.67, 84.45, 81.11, 79.16, 76.47, 75.59, 75.11, 72.96, 72.14, 66.85, 58.54, 52.65, 45.57, 43.22, 35.58, 33.83, 31.91, 29.77, 29.75, 29.53, 29.48, 29.45, 29.33, 29.21, 27.23, 27.20, 26.85, 23.96, 23.84, 22.73, 22.69, 22.18, 14.81, 9.62. HRMS (ESI): m/z (M+Na)⁺ calcd. for C₆₉H₈₉NO₁₈Na, 1242.5977; found, 1242.5965.

(2aR,4S,4aS,6R,9S,11S,12S,12aR,12bS)-9-(((2R,3S)-3-benzamido-2-(((((4R)-2-((Z)-heptadec-8-en-1-yl)-1,3-dioxolan- 4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyl diacetate (NCP-124)

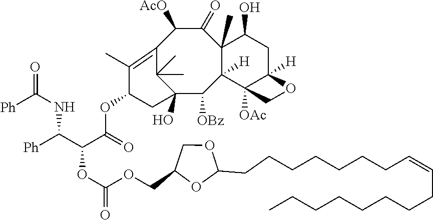

[α]_{D}²⁰=−46.6; (c2, CHCl₃). ¹H NMR (400 MHz, CDCl₃) δ 8.16-8.13; (m, 2H), 7.74; (dd, J=8.0, 1.6 Hz, 2H), 7.62-7.60; (m, 1H), 7.54-7.48; (m, 3H), 7.45-7.34; (m, 7H), 6.92; (dd, J=9.4, 2.6 Hz, 1H), 6.31-6.27; (m, 2H), 6.00; (dd, J=9.4, 2.5 Hz, 1H), 5.69; (d, J=7.1 Hz, 1H), 5.45-5.41; (m, 1H), 5.35-5.33; (m, 2H), 4.99-4.96; (m, 1H), 4.88; (t, J=4.8 Hz, 1H), 4.47-4.41; (m, 1H), 4.32; (d, J=8.8 Hz, 1H), 4.29-4.24; (m, 1H), 4.23-4.18; (m, 2H), 4.15-4.08; (m, 2H), 3.90; (dd, J=8.6, 6.9 Hz, 0.72H), 3.84-3.76; (m, 2H), 3.57; (dd, J=8.6, 6.8 Hz, 0.36H), 2.60-2.53; (m, 1H), 2.50; (d, J=4.1 Hz, 1H), 2.46; (s, 3H), 2.43-2.38; (m, 1H), 2.23; (s, 3H), 2.04; (s, 1H), 2.03-1.97; (m, 4H), 1.94; (d, J=1.6 Hz, 3H), 1.90-1.85; (m, 1H), 1.84; (s, 1H), 1.72; (s, 2H), 1.68; (s, 2H), 1.65-1.63; (m, 1H), 1.41-1.37; (m, 2H), 1.32-1.22; (m, 22H), 1.14; (s, 3H), 0.88; (t, J=6.7 Hz, 3H). ¹³C NMR (101 MHz, CDCl₃) δ 203.79, 171.26, 169.86, 167.77, 167.16, 167.03, 154.03, 142.60, 136.64, 133.68, 133.47, 132.89, 132.08, 130.24, 129.98, 129.78, 129.19, 129.15, 128.75, 128.72, 128.56, 127.16, 126.54, 105.71, 105.02, 84.45, 81.11, 79.14, 75.59, 75.11, 72.96, 72.14, 69.12, 66.93, 60.41, 58.54, 52.63, 45.57, 43.22, 35.59, 33.86, 33.83, 31.91, 29.77, 29.75, 29.53, 29.47, 29.44, 29.32, 29.21, 27.23, 27.20, 26.84, 23.96, 23.86, 22.73, 22.69, 22.18, 21.06, 14.82, 9.62. HRMS (ESI): m/z (M+Na)⁺ calcd. for C₆₉H₈₉NO₁₈Na, 1242.5977; found, 1242.5964.

(2aR,4S,4aS,6R,9S,11S,12S,12aR,12bS)-9-(((2R,3S)-3-benzamido-2-(((((4S)-2-((Z)-heptadec-8-en-1-yl)-1,3-dioxolan- 4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyl diacetate (NCP-125).

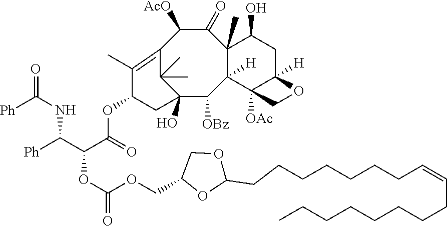

[α]_{D}²⁰=−48.8; (c2, CDCl₃). ¹H NMR (400 MHz, CDCl₃) δ 8.16-8.13; (m, 2H), 7.75-7.73; (m, 2H), 7.63-7.58; (m, 1H), 7.54-7.48; (m, 3H), 7.45-7.34; (m, 7H), 6.93; (dd, J=9.4, 4.9 Hz, 1H), 6.29; (s, 2H), 6.00; (dd, J=9.4, 2.5 Hz, 1H), 5.69; (d, J=7.1 Hz, 1H), 5.42; (d, J=2.6 Hz, 1H), 5.36-5.33; (m, 2H), 4.99-4.93; (m, 1H), 4.87; (t, J=4.8 Hz, 1H), 4.47-4.41; (m, 1H), 4.32; (d, J=8.6 Hz, 1H), 4.30-4.23; (m, 1H), 4.23-4.19; (m, 2H), 4.15; (dd, J=5.5, 2.1 Hz, 1H), 4.13-4.09; (m, 1H), 3.90; (dd, J=8.6, 6.9 Hz, 0.73H), 3.84-3.78; (m, 2H), 3.61; (dd, J=8.6, 6.8 Hz, 0.36H), 2.60-2.52; (m, 1H), 2.50; (dd, J=4.2, 1.7 Hz, 1H), 2.46; (d, J=1.8 Hz, 3H), 2.44-2.38; (m, 1H), 2.23; (s, 3H), 2.04; (s, 1H), 2.00; (q, J=6.5 Hz, 4H), 1.93; (s, 3H), 1.90-1.85; (m, 1H), 1.83; (s, 1H), 1.69; (s, 5H), 1.65-1.61; (m, 1H), 1.40-1.33; (m, 2H), 1.28-1.26; (m, 21H), 1.14; (s, 3H), 0.90-0.85; (m, 3H). ¹³C NMR (101 MHz, CDCl₃) δ 203.78, 171.23, 169.85, 167.77, 167.74, 167.20, 167.00, 154.09, 154.03, 142.57, 136.66, 133.66, 133.47, 132.90, 132.07, 130.23, 129.98, 129.78, 129.20, 129.14, 128.75, 128.71, 128.56, 127.18, 126.56, 105.66, 105.08, 84.45, 81.10, 79.12, 75.59, 75.12, 72.88, 72.82, 72.14, 68.96, 68.32, 66.84, 60.41, 58.52, 52.68, 45.58, 43.22, 35.61, 35.55, 33.87, 33.82, 31.91, 31.59, 29.77, 29.75, 29.52, 29.47, 29.44, 29.32, 29.21, 27.23, 27.20, 26.84, 23.96, 23.84, 22.73, 22.69, 22.19, 21.05, 14.79, 14.20, 9.63. HRMS (ESI): m/z (M+Na)⁺ calcd. for C₆₉H₈₉NO₁₈Na, 1242.5977; found, 1242.5964.

(2aR,4S,4aS,6R,9S,11S,12S,12aR,12bS)-9-(((2R,3S)-3-benzamido-2-(((((2R,4R)-2-((Z)-heptadec-8-en-1-yl)-1,3- dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyl diacetate (NCP-127).

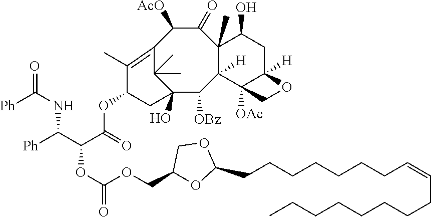

¹H NMR (500 MHz, CDCl₃) δ 8.15-8.13; (m, 2H), 7.75-7.73; (m, 2H), 7.62-7.59; (m, 1H), 7.51; (q, J=7.4 Hz, 3H), 7.43-7.36; (m, 7H), 6.92; (d, J=9.3 Hz, 1H), 6.29; (d, J=2.5 Hz, 2H), 6.00; (dd, J=9.4, 2.6 Hz, 1H), 5.69; (d, J=7.1 Hz, 1H), 5.43; (d, J=2.6 Hz, 1H), 5.35-5.32; (m, 2H), 4.98; (d, J=3.1 Hz, 1H), 4.96; (d, J=4.1 Hz, 1H), 4.46-4.42; (m, 1H), 4.33-4.30; (m, 2H), 4.23-4.17; (m, 3H), 4.15-4.10; (m, 2H), 3.81; (d, J=7.0 Hz, 1H), 3.57; (dd, J=8.7, 6.7 Hz, 1H), 2.60-2.54; (m, 1H), 2.48; (d, J=8.3 Hz, 4H), 2.42-2.37; (m, 1H), 2.23; (s, 3H), 2.21-2.18; (m, 1H), 2.04; (s, 1H), 2.01-1.98; (m, 3H), 1.93; (d, J=1.4 Hz, 3H), 1.90-1.85; (m, 1H), 1.81; (s, 1H), 1.69; (d, J=9.1 Hz, 4H), 1.38; (dd, J=10.0, 5.0 Hz, 2H), 1.31-1.24; (m, 23H), 1.14; (s, 3H), 0.87; (t, J=6.8 Hz, 3H). ¹³C NMR (126 MHz, CDCl₃) δ 203.79, 177.71, 171.28, 169.86, 167.77, 167.13, 167.04, 154.11, 142.62, 136.63, 133.70, 133.47, 132.87, 132.08, 130.24, 129.98, 129.78, 129.17, 129.15, 128.76, 128.72, 128.56, 127.17, 126.55, 105.01, 84.44, 81.09, 79.15, 75.59, 75.09, 73.02, 72.16, 72.12, 68.51, 68.45, 66.81, 58.53, 52.63, 45.56, 43.21, 35.56, 35.53, 33.85, 31.91, 29.77, 29.75, 29.53, 29.51, 29.44, 29.32, 29.21, 27.80, 27.22, 27.20, 26.84, 23.86, 22.73, 22.69, 22.19, 14.82, 9.61. HRMS (ESI): m/z (M+Na)⁺ calcd. for C₆₉H₈₉NO₁₈Na, 1242.5977; found, 1242.5973.

(2aR,4S,4aS,6R,9S,11S,12S,12aR,12bS)-9-(((2R,3S)-3-benzamido-2-(((((2S,4R)-2-((Z)-heptadec-8-en-1-yl)-1,3- dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyldiacetate (NCP-128).

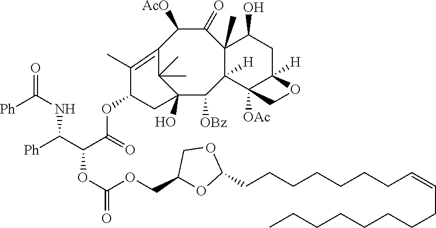

¹H NMR (400 MHz, CDCl₃) δ 8.16-8.11; (m, 2H), 7.76-7.71; (m, 2H), 7.63-7.56; (m, 1H), 7.50; (td, J=7.5, 4.4 Hz, 3H), 7.45-7.35; (m, 7H), 6.95; (d, J=9.3 Hz, 1H), 6.32-6.26; (m, 2H), 6.00; (dd, J=9.4, 2.5 Hz, 1H), 5.69; (d, J=7.1 Hz, 1H), 5.43; (d, J=2.6 Hz, 1H), 5.35; (td, J=7.0, 5.9, 4.0 Hz, 2H), 4.97; (dd, J=9.7, 2.3 Hz, 1H), 4.88; (t, J=4.8 Hz, 1H), 4.44; (ddd, J=10.6, 6.5, 3.2 Hz, 1H), 4.31; (d, J=8.6 Hz, 1H), 4.26; (td, J=6.6, 3.1 Hz, 1H), 4.23-4.17; (m, 2H), 4.11; (dd, J=11.0, 6.3 Hz, 1H), 3.90; (dd, J=8.7, 6.8 Hz, 1H), 3.84-3.76; (m, 2H), 2.54; (dq, J=14.5, 6.1, 4.7 Hz, 2H), 2.46; (s, 3H), 2.43-2.36; (m, 1H), 2.22; (s, 4H), 2.01; (q, J=7.1, 6.4 Hz, 4H), 1.93; (d, J=1.5 Hz, 3H), 1.89; (d, J=6.0 Hz, 2H), 1.68; (s, 3H), 1.66; (d, J=4.4 Hz, 1H), 1.35-1.22; (m, 26H), 1.14; (s, 3H), 0.88; (t, J=6.7 Hz, 3H). ¹³C NMR (101 MHz, CDCl₃) δ 203.78, 171.24, 169.85, 167.78, 167.22, 167.01, 154.04, 142.57, 136.64, 133.67, 133.47, 132.91, 132.08, 130.24, 129.98, 129.78, 129.20, 129.15, 128.75, 128.72, 128.56, 127.17, 126.56, 115.49, 105.71, 84.45, 81.10, 79.13, 75.59, 75.13, 72.97, 72.14, 69.13, 66.93, 58.52, 52.65, 45.59, 43.22, 35.60, 35.55, 33.83, 31.91, 29.77, 29.75, 29.71, 29.53, 29.47, 29.44, 29.33, 29.21, 27.23, 27.20, 26.84, 23.95, 22.73, 22.69, 22.18, 14.82, 14.21, 9.63. HRMS (ESI): m/z (M+Na)⁺ calcd. for C₆₉H₈₉NO₁₈Na, 1242.5977; found, 1242.5972.

(2aR,4S,4aS,6R,9S,11S,12S,12aR,12bS)-9-(((2R,3S)-3-benzamido-2-(((((2R,4S)-2-((Z)-heptadec-8-en-1-yl)-1,3- dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyl diacetate (NCP-129).

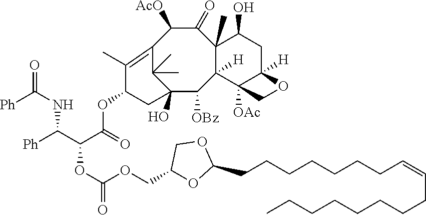

¹H NMR (500 MHz, CDCl₃) δ 8.15; (d, J=7.7 Hz, 2H), 7.74; (d, J=7.7 Hz, 2H), 7.61; (t, J=7.4 Hz, 1H), 7.51; (q, J=7.2 Hz, 3H), 7.45-7.35; (m, 7H), 6.91; (d, J=9.3 Hz, 1H), 6.31; (d, J=10.3 Hz, 2H), 6.00; (dd, J=9.2, 2.4 Hz, 1H), 5.69; (d, J=7.1 Hz, 1H), 5.42; (d, J=2.5 Hz, 1H), 5.37-5.30; (m, 2H), 4.98; (d, J=9.3 Hz, 1H), 4.94; (t, J=4.8 Hz, 1H), 4.46-4.42; (m, 1H), 4.34-4.28; (m, 2H), 4.21; (t, J=4.3 Hz, 3H), 4.15-4.11; (m, 2H), 3.82; (d, J=6.9 Hz, 1H), 3.61; (t, J=7.7 Hz, 1H), 2.59-2.53; (m, 1H), 2.49; (d, J=4.0 Hz, 1H), 2.47; (s, 2H), 2.41; (dd, J=15.4, 9.3 Hz, 2H), 2.23; (s, 3H), 2.04; (s, 1H), 2.03-1.96; (m, 4H), 1.93; (s, 3H), 1.91-1.86; (m, 1H), 1.77; (d, J=2.9 Hz, 1H), 1.67; (d, J=12.1 Hz, 5H), 1.65-1.62; (m, 1H), 1.33-1.23; (m, 23H), 1.14; (s, 3H), 0.88; (t, J=6.7 Hz, 3H). ¹³C NMR (126 MHz, CDCl₃) δ 203.79, 171.28, 169.85, 167.73, 167.12, 167.06, 154.09, 142.63, 136.67, 133.70, 133.46, 132.87, 132.09, 130.25, 129.99, 129.78, 129.16, 129.14, 128.77, 128.73, 128.56, 127.17, 126.55, 105.09, 84.45, 81.10, 79.18, 75.59, 75.09, 72.87, 72.16, 72.12, 68.31, 66.69, 60.42, 58.54, 52.66, 45.57, 43.21, 35.58, 35.53, 33.87, 31.91, 29.78, 29.76, 29.53, 29.52, 29.45, 29.33, 29.22, 27.23, 27.21, 26.85, 23.84, 22.75, 22.70, 22.18, 14.82, 14.22, 9.62. HRMS (ESI): m/z (M+Na)⁺ calcd. for C₆₉H₈₉NO₁₈Na, 1242.5977; found,1242.5978.

(2aR,4S,4aS,6R,9S,11S,12S,12aR,12bS)-9-(((2R,3S)-3-benzamido-2-(((((2S,4S)-2-((Z)-heptadec-8-en-1-yl)-1,3- dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11- methanocyclodeca[3,4]benzo[1,2-b]oxete-6,12b(2aH)-diyl diacetate (NCP-130).

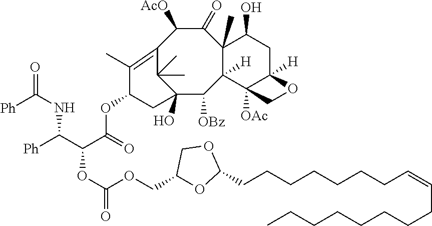

¹H NMR (400 MHz, CDCl₃) δ 8.14; (d, J=7.7 Hz, 2H), 7.74; (d, J=7.7 Hz, 2H), 7.60; (t, J=7.4 Hz, 1H), 7.54-7.46; (m, 3H), 7.44-7.35; (m, 7H), 6.92; (d, J=9.3 Hz, 1H), 6.28; (d, J=8.1 Hz, 2H), 6.00; (dd, J=9.3, 2.5 Hz, 1H), 5.69; (d, J=7.1 Hz, 1H), 5.42; (d, J=2.6 Hz, 1H), 5.35-5.32; (m, 2H), 5.01-4.94; (m, 1H), 4.87; (t, J=4.8 Hz, 1H), 4.47-4.42; (m, 1H), 4.32; (d, J=8.5 Hz, 1H), 4.29-4.23; (m, 1H), 4.21; (d, J=8.3 Hz, 1H), 4.15; (dd, J=5.5, 2.1 Hz, 2H), 4.11; (t, J=7.1 Hz, 2H), 3.90; (t, J=7.8 Hz, 1H), 3.83-3.80; (m, 1H), 2.58-2.56; (m, 1H), 2.49; (d, J=4.1 Hz, 1H), 2.46; (s, 3H), 2.44-2.36; (m, 1H), 2.23; (s, 3H), 2.04; (s, 1H), 2.00; (q, J=6.5 Hz, 4H), 1.93; (s, 3H), 1.90-1.84; (m, 1H), 1.77; (s, 1H), 1.69; (d, J=5.5 Hz, 5H), 1.65-1.63; (m, 1H), 1.31-1.22; (m, 23H), 1.14; (s, 3H), 0.87; (t, J=6.6 Hz, 3H). ¹³C NMR (101 MHz, CDCl₃) δ 203.79, 171.26, 169.85, 167.75, 167.11, 167.05, 154.03, 142.64, 136.68, 133.68, 133.48, 132.87, 132.07, 130.24, 129.99, 129.78, 129.18, 129.14, 128.76, 128.71, 128.55, 127.17, 126.56, 105.67, 84.45, 81.10, 79.19, 75.59, 75.12, 72.82, 72.14, 68.96, 66.86, 60.41, 58.54, 52.67, 45.57, 43.21, 35.60, 35.54, 33.84, 31.91, 29.78, 29.75, 29.53, 29.47, 29.45, 29.33, 29.22, 27.23, 27.20, 26.85, 23.97, 22.74, 22.69, 22.19, 21.06, 14.81, 14.21, 9.62. HRMS (ESI): m/z (M+Na)⁺ calcd. for C₆₉H₈₉NO₁₈Na, 1242.5977; found, 1242.5972.

(2aR,4S,4aS,6R,9S,11S,12S,12aR,12bS)-9-(((2R,3S)-3-benzamido-2-((((2,2-dimethyl-1,3-dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-126).

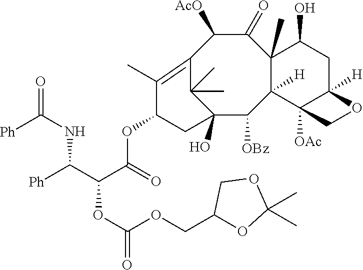

¹H NMR (500 MHz, CDCl₃) δ 8.15-8.13; (m, 2H), 7.74-7.72; (m, 2H), 7.62-7.58; (m, 1H), 7.53-7.48; (m, 3H), 7.44-7.35; (m, 7H), 6.95; (d, J=9.3 Hz, 1H), 6.32-6.24; (m, 2H), 6.00; (dd, J=9.5, 2.6 Hz, 1H), 5.69; (d, J=7.1 Hz, 1H), 5.42; (d, J=2.6 Hz, 1H), 4.97; (dd, J=9.7, 2.3 Hz, 1H), 4.46-4.41; (m, 1H), 4.33-4.28; (m, 2H), 4.25-4.19; (m, 2H), 4.17-4.12; (m, 1H), 4.09-4.04; (m, 1H), 3.81; (d, J=7.0 Hz, 1H), 3.76; (dd, J=8.7, 5.8 Hz, 1H), 2.60-2.53; (m, 1H), 2.52; (d, J=4.0 Hz, 1H), 2.46; (s, 3H), 2.41; (dd, J=15.4, 9.4 Hz, 1H), 2.22; (s, 3H), 1.93; (d, J=1.4 Hz, 3H), 1.90; (s, 1H), 1.89-1.85; (m, 1H), 1.82; (s, 1H), 1.68; (s, 3H), 1.36; (d, J=10.3 Hz, 6H), 1.24; (s, 3H), 1.13; (s, 3H). ¹³C NMR (126 MHz, CDCl₃) δ 203.79, 171.25, 169.85, 167.77, 167.19, 167.01, 154.10, 142.59, 136.66, 133.67, 133.49, 132.89, 132.07, 130.23, 129.21, 129.13, 128.75, 128.71, 128.54, 127.17, 126.54, 115.60, 110.09, 109.99, 84.45, 81.10, 79.12, 75.59, 75.12, 73.08, 72.13, 68.68, 65.91, 58.52, 52.66, 45.58, 43.21, 35.61, 35.55, 26.84, 26.56, 25.32, 22.73, 22.19, 20.83, 14.79, 9.63. HRMS (ESI): m/z (M+Na)⁺ calcd. for C₅₄H₆₁NO₁₈Na, 1034.3786; found, 1034.3776.

(2aR,4S,4aS,6R,9S,11S,12S,12aR,12bS)-9-(((2R,3S)-3-benzamido-2-(((((S)-2,2-dimethyl-1,3-dioxolan-4-yl)methoxy)carbonyl)oxy)- 3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-131).

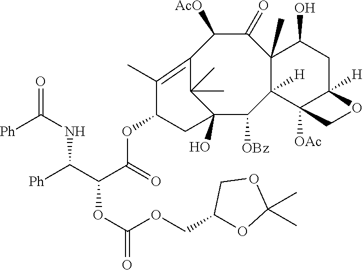

¹H NMR (500 MHz, CDCl₃) δ 8.16-8.10; (m, 2H), 7.76-7.70; (m, 2H), 7.62-7.57; (m, 1H), 7.53-7.47; (m, 3H), 7.44-7.35; (m, 7H), 6.95; (d, J=9.3 Hz, 1H), 6.32-6.24; (m, 2H), 6.00; (dd, J=9.5, 2.6 Hz, 1H), 5.69; (d, J=7.1 Hz, 1H), 5.42; (d, J=2.6 Hz, 1H), 4.97; (dd, J=9.7, 2.3 Hz, 1H), 4.43; (ddd, J=10.9, 6.6, 4.0 Hz, 1H), 4.33-4.28; (m, 2H), 4.25-4.19; (m, 2H), 4.17-4.12; (m, 1H), 4.09-4.04; (m, 1H), 3.81; (d, J=7.0 Hz, 1H), 3.76; (dd, J=8.7, 5.8 Hz, 1H), 2.60-2.53; (m, 1H), 2.52; (d, J=4.0 Hz, 1H), 2.46; (s, 3H), 2.41; (dd, J=15.4, 9.4 Hz, 1H), 2.22; (s, 3H), 1.93; (d, J=1.4 Hz, 3H), 1.90; (s, 1H), 1.89-1.85; (m, 1H), 1.82; (s, 1H), 1.68; (s, 3H), 1.37; (s, 3H), 1.35; (s, 3H), 1.24; (s, 3H), 1.13; (s, 3H). ¹³C NMR (126 MHz, CDCl₃) δ 203.79, 171.25, 169.85, 167.77, 167.19, 167.01, 154.10, 142.59, 136.66, 133.67, 133.49, 132.89, 132.07, 130.23, 129.21, 129.13, 128.75, 128.71, 128.54, 127.17, 126.54, 115.60, 110.09, 109.99, 84.45, 81.10, 79.12, 75.59, 75.12, 73.08, 72.13, 68.68, 65.91, 58.52, 52.66, 45.58, 43.21, 35.61, 35.55, 26.84, 26.56, 25.32, 22.73, 22.19, 20.83, 14.79, 9.63. HRMS (ESI): m/z (M+Na)⁺ calcd. for C₅₄H₆₁NO₁₈Na, 1034.3786; found, 1034.3783.

(2aR,4S,4aS,6R,9S,11S,12S,12aR,12bS)-9-(((2R,3S)-3-benzamido-2-(((((R)-2,2-dimethyl-1,3-dioxolan-4-yl)methoxy)carbonyl)oxy)- 3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-132).

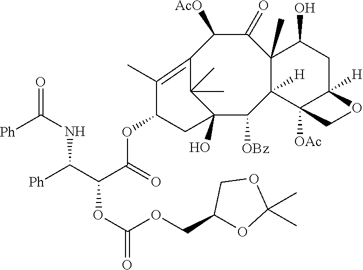

¹H NMR (400 MHz, CDCl₃) δ 8.17-8.10; (m, 2H), 7.77-7.70; (m, 2H), 7.60; (t, J=7.4 Hz, 1H), 7.54-7.47; (m, 3H), 7.45-7.34; (m, 7H), 6.93; (d, J=9.3 Hz, 1H), 6.30; (d, J=4.6 Hz, 2H), 6.00; (dd, J=9.3, 2.3 Hz, 1H), 5.69; (d, J=7.1 Hz, 1H), 5.44; (d, J=2.6 Hz, 1H), 4.96; (d, J=8.0 Hz, 1H), 4.43; (dt, J=10.8, 5.3 Hz, 1H), 4.35-4.27; (m, 2H), 4.27-4.18; (m, 2H), 4.16-4.12; (m, 1H), 4.06; (dd, J=8.7, 6.5 Hz, 1H), 3.81; (d, J=7.0 Hz, 1H), 3.73; (dd, J=8.7, 5.7 Hz, 1H), 2.59-2.49; (m, 2H), 2.45; (s, 3H), 2.42-2.36; (m, 1H), 2.22; (s, 3H), 1.93; (s, 3H), 1.88; (s, 2H), 1.84; (s, 1H), 1.68; (s, 3H), 1.41; (s, 3H), 1.35; (s, 3H), 1.24; (s, 3H), 1.13; (s, 3H). ¹³C NMR (101 MHz, CDCl₃) δ 203.77, 171.21, 169.85, 167.75, 167.14, 167.01, 154.09, 142.56, 136.66, 133.65, 133.51, 132.91, 132.05, 130.23, 129.22, 129.14, 128.74, 128.70, 128.54, 127.16, 126.56, 110.14, 84.45, 81.11, 79.14, 76.46, 75.59, 75.14, 73.17, 72.12, 69.07, 66.10, 58.53, 52.65, 45.60, 43.21, 35.56, 26.84, 26.66, 25.28, 22.72, 22.16, 20.82, 14.79, 9.62. HRMS (ESI): m/z (M+Na)⁺ calcd. for C₅₄H₆₁NO₁₈Na, 1034.3786; found, 1034.3791.

NCC-SA-01

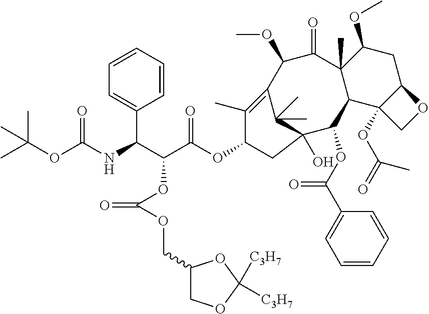

(2aR,4S,4aS,6R,9S,11S,12S,12aR,12bS)-12b-acetoxy-9-(((2R,3S)-3-((tert-butoxycarbonyl)amino)-2-((((2,2-dipropyl-1,3- dioxolan-4-yl)methoxy)carbonyl)oxy)-3-phenylpropanoyl)oxy)-11-hydroxy-4,6-dimethoxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-12-yl benzoate: ¹H NMR (500 MHz, Chloroform-d) δ 8.13-8.08; (m, 2H), 7.63-7.57; (m, 1H), 7.49; (t, J=7.8 Hz, 2H), 7.44-7.37; (m, 2H), 7.37-7.29; (m, 3H), 6.29; (t, J=9.1 Hz, 1H), 5.65; (d, J=7.0 Hz, 1H), 5.46; (s, 1H), 5.40; (s, 1H), 5.25; (dd, J=8.9, 2.6 Hz, 1H), 4.99; (dd, J=9.7, 2.1 Hz, 1H), 4.83; (s, 1H), 4.34-4.24; (m, 2H), 4.22-4.10; (m, 3H), 4.06; (ddd, J=8.4, 6.4, 3.6 Hz, 1H), 3.90; (dd, J=10.7, 6.4 Hz, 1H), 3.85; (d, J=6.9 Hz, 1H), 3.68; (ddd, J=17.8, 8.5, 6.5 Hz, 1H), 3.44; (s, 3H), 3.30; (s, 3H), 2.70; (ddd, J=14.1, 9.7, 6.4 Hz, 1H), 2.44; (s, 3H), 2.33; (s, 1H), 2.00; (s, 3H), 1.79; (ddd, J=13.5, 10.6, 2.2 Hz, 1H), 1.72; (s, 3H), 2.36-216; (m, 2H), 1.71; (s, 3H), 1.63-1.52; (m, 4H), 1.37-1.26; (m, 13H), 1.22; (s, 3H), 1.20; (s, 3H), 0.91; (td, J=7.4, 2.3 Hz, 6H). ¹³C NMR (126 MHz, CDCl₃) δ 205.10, 169.88, 168.13, 167.19, 154.32, 135.23, 133.74, 130.32, 129.40, 129.11, 128.80, 128.44, 126.58, 126.56, 113.57, 113.52, 84.32, 82.68, 81.77, 80.84, 80.64, 79.03, 76.63, 74.89, 73.34, 73.23, 72.53, 69.10, 68.83, 66.80, 66.59, 57.32, 57.22, 47.51, 39.72, 39.69, 39.34, 39.32, 35.10, 32.19, 28.27, 26.82, 22.97, 21.17, 17.38, 17.04, 17.02, 14.60, 14.50, 14.49, 10.53. HRMS (ESI): m/z [M+Na]⁺ calcd. for C₅₆H₇₅NO₁₈, 1072.4984; found, 1072.4872.

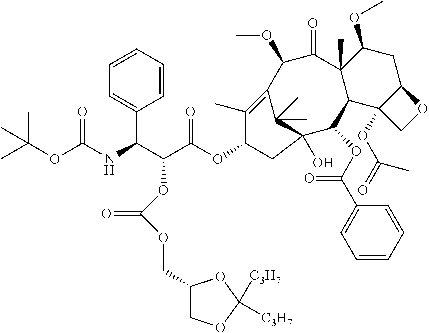

NCC-SA-02-(2aR,4S,4aS,6R,9S,11S,12S,12aR,12bS)-12b-acetoxy-9-(((2R,3S)-3-((tert-butoxycarbonyl)amino)-2-(((((R)-2,2- dipropyl-1,3-dioxolan-4-yl)methoxy)carbonyl)oxy)-3-phenylpropanoyl)oxy)-11-hydroxy-4,6-dimethoxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-12-yl benzoate: ¹H NMR (500 MHz, CDCl₃) δ 8.14-8.08; (m, 2H), 7.64-7.56; (m, 1H), 7.49; (t, J=7.8 Hz, 2H), 7.40; (tt, J=7.0, 0.9 Hz, 2H), 7.37-7.29; (m, 3H), 6.29; (t, J=9.1 Hz, 1H), 5.65; (d, J=7.0 Hz, 1H), 5.46; (s, 1H), 5.39; (d, J=9.5 Hz, 1H), 5.28-5.22; (m, 1H), 4.99; (dd, J=9.7, 2.1 Hz, 1H), 4.82; (s, 1H), 4.34-4.24; (m, 2H), 4.22-4.15; (m, 2H), 4.14; (dd, J=11.1, 5.2 Hz, 1H), 4.06; (ddd, J=8.4, 6.5, 3.6 Hz, 1H), 3.90; (dd, J=10.7, 6.4 Hz, 1H), 3.85; (d, J=6.9 Hz, 1H), 3.70; (dd, J=8.4, 6.4 Hz, 1H), 3.44; (s, 1H), 3.44; (s, 2H), 3.30; (s, 3H), 2.70; (ddd, J=14.1, 9.8, 6.4 Hz, 1H), 2.44; (s, 3H), 2.36-2.16; (m, 2H), 2.00; (s, 3H), 1.84-1.75; (m, 1H), 1.72; (s, 3H), 1.64-1.51; (m, 4H), 1.41-1.31; (m, 13H), 1.22; (s, 3H), 1.20; (s, 3H), 0.94-0.85; (m, 6H). ¹³C NMR (126 MHz, CDCl₃) δ 205.09, 169.86, 168.13, 167.19, 154.31, 139.55, 135.22, 133.73, 130.31, 129.40, 129.10, 128.79, 128.44, 126.58, 113.57, 84.31, 82.68, 81.77, 80.83, 80.64, 79.03, 76.62, 74.89, 73.23, 72.52, 68.82, 66.59, 57.30, 57.21, 56.98, 47.50, 39.72, 39.69, 39.31, 35.10, 32.18, 28.26, 26.81, 22.97, 21.16, 17.37, 17.02, 14.59, 14.50, 10.52. HRMS (ESI): m/z [M+Na]⁺ calcd. for C₅₆H₇₅NO₁₈, 1072.4984; found, 1072.4867.

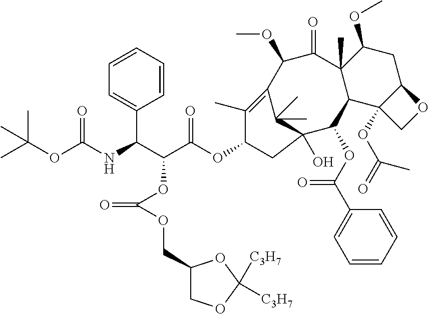

NCC-SA-03-(2aR,4S,4aS,6R,9S,11S,12S,12aR,12bS)-12b-acetoxy-9-(((2R,3S)-3-((tert-butoxycarbonyl)amino)-2-(((((S)-2,2- dipropyl-1,3-dioxolan-4-yl)methoxy)carbonyl)oxy)-3-phenylpropanoyl)oxy)-11-hydroxy-4,6-dimethoxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-12-yl benzoate: ¹H NMR (500 MHz, Chloroform-d) δ 8.14-8.08; (m, 2H), 7.63-7.56; (m, 1H), 7.49; (t, J=7.8 Hz, 2H), 7.41; (dd, J=8.2, 6.8 Hz, 2H), 7.37-7.29; (m, 3H), 7.26; (s, 1H), 6.29; (t, J=9.2 Hz, 1H), 5.65; (d, J=7.0 Hz, 1H), 5.46; (s, 1H), 5.39; (d, J=9.9 Hz, 1H), 5.25; (dd, J=9.1, 2.6 Hz, 1H), 5.02-4.96; (m, 1H), 4.83; (s, 1H), 4.34-4.24; (m, 2H), 4.22-4.10; (m, 3H), 4.06; (ddd, J=10.0, 6.5, 3.6 Hz, 1H), 3.90; (dd, J=10.7, 6.4 Hz, 1H), 3.85; (d, J=6.9 Hz, 1H), 3.68; (ddd, J=17.3, 8.4, 6.5 Hz, 1H), 3.44; (s, 3H), 3.30; (s, 3H), 2.70; (ddd, J=13.9, 9.8, 6.4 Hz, 1H), 2.43; (s, 3H), 2.36-216; (m, 2H), 2.00; (s, 3H), 1.79; (ddd, J=13.8, 10.9, 2.2 Hz, 1H), 1.71; (s, 3H), 1.63-1.52; (m, 4H), 1.42-1.31; (m, 13H), 1.22; (s, 3H), 1.20; (s, 3H), 0.94-0.85; (m, 6H). ¹³C NMR (126 MHz, CDCl₃) δ 205.11, 169.87, 168.13, 167.18, 154.31, 139.54, 135.22, 133.73, 130.31, 129.39, 129.10, 128.79, 128.43, 126.58, 126.56, 126.28, 113.57, 84.31, 82.67, 81.76, 80.83, 80.64, 79.02, 76.62, 74.88, 73.33, 72.52, 69.09, 66.79, 57.30, 57.21, 56.97, 47.50, 43.51, 39.71, 39.33, 39.31, 35.09, 32.17, 28.26, 26.81, 22.96, 21.16, 17.37, 17.03, 17.01, 14.59, 14.48, 10.52. HRMS (ESI): m/z [M+Na]⁺ calcd. for C₅₆H₇₅NO₁₈, 1072.4984; found, 1072.4857.

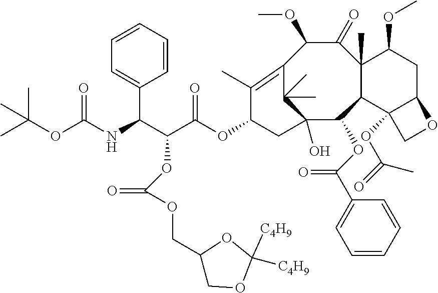

NCC-SA-04-(2aR,4S,4aS,6R,9S,11S,12S,12aR,12bS)-12b-acetoxy-9-(((2R,3S)-3-((tert-butoxycarbonyl)amino)-2-((((2,2-dibutyl-1,3-dioxolan-4-yl)methoxy)carbonyl)oxy)-3-phenylpropanoyl)oxy)-11-hydroxy-4,6-dimethoxy-4a,8,13,13-tetramethyl-5-oxo-2a,3,4,4a,5,6,9,10,11,12,12a,12b- dodecahydro-1H-7,11-methanocyclodeca[3,4]benzo[1,2-b]oxet-12-yl benzoate: ¹H NMR (500 MHz, CDCl₃) δ 8.14-8.08; (m, 2H), 7.64-7.56; (m, 1H), 7.49; (t, J=7.7 Hz, 2H), 7.41; (ddd, J=7.7, 6.5, 1.6 Hz, 2H), 7.37-7.29; (m, 3H), 6.29; (t, J=9.1 Hz, 1H), 5.65; (d, J=7.0 Hz, 1H), 5.46; (s, 1H), 5.40; (s, 1H), 5.25; (dd, J=7.6, 2.6 Hz, 1H), 4.99; (dd, J=9.8, 2.1 Hz, 1H), 4.82; (s, 1H), 4.34-4.25; (m, 2H), 4.23-4.15; (m, 2H), 4.13; (ddd, J=11.2, 5.6, 2.7 Hz, 1H), 4.06; (ddd, J=8.4, 6.5, 3.5 Hz, 1H), 3.90; (dd, J=10.8, 6.4 Hz, 1H), 3.85; (d, J=6.9 Hz, 1H), 3.68; (ddd, J=15.8, 8.4, 6.5 Hz, 1H), 3.44; (d, J=1.0 Hz, 3H), 3.30; (s, 3H), 2.70; (ddd, J=14.2, 9.8, 6.4 Hz, 1H), 2.44; (s, 3H), 2.37-2.17; (m, 2H), 2.00; (d, J=1.5 Hz, 3H), 1.79; (ddd, J=14.3, 10.8, 2.3 Hz, 1H), 1.72; (s, 3H), 1.63-1.53; (m, 4H), 1.35; (s, 9H), 1.34-1.24; (m, 8H), 1.21; (d, J=8.8 Hz, 6H), 0.90; (dtt, J=7.2, 5.3, 2.8 Hz, 6H). ¹³C NMR (126 MHz, CDCl₃) δ 205.10, 169.88, 168.14, 168.12 , 167.18, 154.33, 139.53, 135.23, 133.73, 130.31, 129.40, 129.10, 128.80, 128.43, 126.57, 126.56, 113.74, 113.69, 84.32, 82.67, 81.77, 80.83, 80.64, 79.02, 76.63, 74.89, 73.33, 73.22, 72.53, 69.13, 68.89, 66.80, 66.61, 57.31, 57.29, 57.21, 56.98, 47.50, 43.51, 37.20, 37.18, 36.75, 36.72, 35.10, 32.18, 28.26, 26.81, 26.22, 25.89, 23.10, 23.05, 22.97, 22.52, 21.16, 14.59, 14.20, 10.52. HRMS (ESI): m/z [M+H]⁺ calcd. for C₅₈H₇₉NO₁₈, 1078.5297; found, 1078.5378.

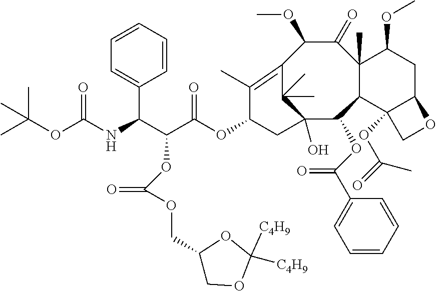

NCC-SA-05-(2aR,4S,4aS,6R,9S,11S,12S,12aR,12bS)-12b-acetoxy-9-(((2R,3S)-3-((tert-butoxycarbonyl)amino)-2-(((((R)-2,2-dibutyl- 1,3-dioxolan-4-yl)methoxy)carbonyl)oxy)-3-phenylpropanoyl)oxy)-11-hydroxy-4,6-dimethoxy-4a,8,13,13-tetramethyl-5-oxo-2a,3,4,4a,5,6,9,10,11,12,12a,12b- dodecahydro-1H-7,11-methanocyclodeca[3,4]benzo[1,2-b]oxet-12-yl benzoate: ¹H NMR (500 MHz, CDCl₃) δ 8.11; (dd, J=8.3, 1.3 Hz, 2H), 7.63-7.56; (m, 1H), 7.49; (t, J=7.7 Hz, 2H), 7.44-7.37; (m, 2H), 7.36-7.29; (m, 3H), 6.29; (t, J=9.1 Hz, 1H), 5.65; (d, J=7.0 Hz, 1H), 5.46; (s, 1H), 5.40; (d, J=9.3 Hz, 1H), 5.28-5.22; (m, 1H), 4.99; (dd, J=9.7, 2.1 Hz, 1H), 4.82; (s, 1H), 4.34-4.24; (m, 2H), 4.23-4.13; (m, 2H), 4.16-4.10; (m, 1H), 4.06; (ddd, J=8.4, 6.4, 3.5 Hz, 1H), 3.90; (dd, J=10.7, 6.4 Hz, 1H), 3.85; (d, J=6.9 Hz, 1H), 3.69; (dd, J=8.4, 6.5 Hz, 1H), 3.44; (d, J=1.0 Hz, 3H), 3.30; (s, 3H), 2.70; (ddd, J=14.1, 9.8, 6.4 Hz, 1H), 2.44; (s, 3H), 2.37-2.20; (m, 2H), 2.00; (d, J=1.4 Hz, 3H), 1.79; (ddd, J=14.0, 11.0, 2.2 Hz, 1H), 1.64-1.53; (m, 4H), 1.35; (s, 9H), 1.34-1.24; (m, 8H), 1.22; (s, 3H), 1.20; (s, 3H), 0.89; (qt, J=5.0, 2.8 Hz, 6H). ¹³C NMR (126 MHz, CDCl₃) δ 205.10, 169.87, 168.12, 167.17, 154.32, 139.55, 135.21, 133.73, 130.31, 129.40, 129.09, 128.79, 128.43, 126.57, 113.69, 84.31, 82.67, 81.76, 80.83, 80.64, 79.01, 76.62, 74.88, 73.32, 73.22, 72.52, 68.88, 66.61, 57.28, 57.20, 56.97, 47.50, 43.50, 37.17, 36.71, 35.10, 32.18, 28.26, 26.80, 26.21, 25.88, 23.10, 23.04, 22.96, 21.16, 14.58, 14.20, 10.52. HRMS (ESI): m/z [M+Na]⁺ calcd. for C₅₈H₇₉NO₁₈, 1100.5297; found, 1100.5176.

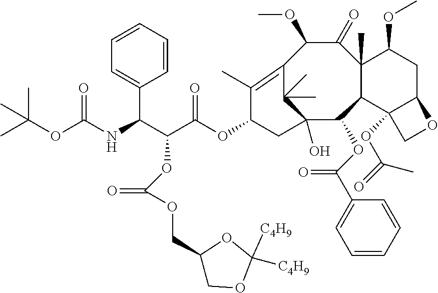

NCC-SA-06-(2aR,4S,4aS,6R,9S,11S,12S,12aR,12bS)-12b-acetoxy-9-(((2R,3S)-3-((tert-butoxycarbonyl)amino)-2-(((((S)-2,2-dibutyl- 1,3-dioxolan-4-yl)methoxy)carbonyl)oxy)-3-phenylpropanoyl)oxy)-11-hydroxy-4,6-dimethoxy-4a,8,13,13-tetramethyl-5-oxo-2a,3,4,4a,5,6,9,10,11,12,12a,12b- dodecahydro-1H-7,11-methanocyclodeca[3,4]benzo[1,2-b]oxet-12-yl benzoate: ¹H NMR (500 MHz, CDCl₃) δ 8.14-8.08; (m, 2H), 7.64-7.56; (m, 1H), 7.49; (t, J=7.8 Hz, 2H), 7.41; (dd, J=8.2, 6.8 Hz, 2H), 7.37-7.29; (m, 3H), 6.29; (t, J=9.2 Hz, 1H), 5.65; (d, J=7.0 Hz, 1H), 5.46; (s, 1H), 5.39; (d, J=10.5 Hz, 1H), 5.26; (d, J=2.5 Hz, 1H), 4.99; (dd, J=9.7, 2.1 Hz, 1H), 4.82; (s, 1H), 4.34-4.25; (m, 2H), 4.23-4.09; (m, 3H), 4.12-4.03; (m, 1H), 3.90; (dd, J=10.7, 6.4 Hz, 1H), 3.85; (d, J=6.9 Hz, 1H), 3.66; (dd, J=8.5, 6.6 Hz, 1H), 3.44; (s, 3H), 3.30; (s, 3H), 2.70; (ddd, J=14.2, 9.8, 6.4 Hz, 1H), 2.43; (s, 3H), 2.37-2.16; (m, 2H), (s, 1H), 2.00; (s, 3H), 1.79; (ddd, J=13.4, 10.8, 2.2 Hz, 1H), 1.71; (s, 3H), 1.63-1.55; (m, 4H), 1.34; (s, 9H), 1.34-1.23; (m, 8H), 1.22; (s, 3H), 1.20; (s, 3H), 0.89; (td, J=7.1, 2.2 Hz, 6H). ¹³C NMR (126 MHz, CDCl₃) δ 205.12, 169.89, 168.14, 167.19, 154.33, 139.53, 135.23, 133.74, 130.32, 129.40, 129.11, 128.80, 128.44, 126.56, 113.74, 84.32, 82.68, 81.77, 80.83, 80.65, 79.02, 76.63, 74.89, 73.33, 72.53, 69.13, 66.80, 57.30, 57.21, 56.98, 47.50, 43.51, 37.20, 36.75, 35.10, 32.18, 28.26, 26.81, 26.22, 25.89, 23.11, 23.04, 22.97, 21.16, 14.59, 14.20, 10.53. HRMS (ESI): m/z [M+H]⁺ calcd. for C₅₈H₇₉NO₁₈, 1078.5297; found, 1078.5388.

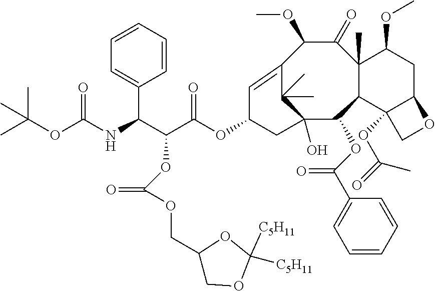

NCC-SA-07-(2aR,4S,4aS,6R,9S,11S,12S,12aR,12bS)-12b-acetoxy-9-(((2R,3S)-3-((tert-butoxycarbonyl)amino)-2-((((2,2-dipentyl-1,3-dioxolan-4-yl)methoxy)carbonyl)oxy)-3-phenylpropanoyl)oxy)-11-hydroxy-4,6-dimethoxy-4a,8,13,13-tetramethyl-5-oxo-2a,3,4,4a,5,6,9,10,11,12,12a,12b- dodecahydro-1H-7,11-methanocyclodeca[3,4]benzo[1,2-b]oxet-12-yl benzoate: ¹H NMR (500 MHz, CDCl₃) δ 8.12-8.09; (m, 2H), 7.65-7.56; (m, 1H), 7.49; (t, J=7.8 Hz, 2H), 7.44-7.38; (m, 2H), 7.36-7.30; (m, 3H), 6.29; (t, J=9.2 Hz, 1H), 5.65; (d, J=7.0 Hz, 1H), 5.46; (s, 1H), 5.42-5.35; (m, 1H), 5.25; (dd, J=6.7, 2.6 Hz, 1H), 4.99; (dd, J=9.8, 2.1 Hz, 1H), 4.83; (s, 1H), 4.36-4.25; (m, 2H), 4.23-4.10; (m, 3H), 4.06; (ddd, J=8.4, 6.5, 3.4 Hz, 1H), 3.90; (dd, J=10.7, 6.4 Hz, 1H), 3.85; (d, J=7.0 Hz, 1H), 3.68; (ddd, J=17.0, 8.4, 6.5 Hz, 1H), 3.44; (d, J=1.0 Hz, 3H), 3.30; (s, 3H), 2.70; (ddd, J=14.1, 9.7, 6.4 Hz, 1H), 2.44; (s, 3H), 2.36-2.16; (m, 2H), 2.00; (s, 3H), 1.79; (ddd, J=13.8, 11.0, 2.2 Hz, 1H), 1.72; (s, 3H), 1.67-1.53; (m, 4H), 1.34; (s, 9H), 1.34-1.23; (m, 12H), 1.22; (s, 3H), 1.20; (s, 3H), 0.88; (td, J=7.1, 2.2 Hz, 6H). ¹³C NMR (126 MHz, CDCl₃) δ 205.15, 169.88, 168.14, 168.12, 167.19, 154.32, 139.56, 135.22, 133.74, 130.31, 129.39, 129.11, 128.80, 128.44, 126.57, 126.56, 126.31, 113.76, 113.70, 84.32, 82.67, 81.77, 80.83, 80.66, 79.02, 76.63, 74.88, 73.32, 73.20, 72.52, 69.18, 68.92, 66.82, 66.64, 57.29, 57.21, 56.98, 47.50, 43.51, 37.43, 37.40, 36.96, 36.92, 35.10, 32.21, 32.18, 32.16, 32.15, 28.26, 26.81, 23.74, 23.42, 23.41, 22.96, 22.75, 22.73, 21.16, 14.58, 14.19, 14.17, 10.53. HRMS (ESI): m/z [M+H]⁺ calcd. for C₆₀H₈₄NO₁₈, 1106.5610; found, 1106.5680.

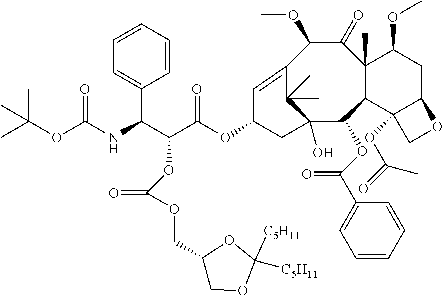

NCC-SA-08-(2aR,4S,4aS,6R,9S,11S,12S,12aR,12bS)-12b-acetoxy-9-(((2R,3S)-3-((tert-butoxycarbonyl)amino)-2-(((((R)-2,2- dipentyl-1,3-dioxolan-4-yl)methoxy)carbonyl)oxy)-3-phenylpropanoyl)oxy)-11-hydroxy-4,6-dimethoxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-12-yl benzoate: ¹H NMR (500 MHz, CDCl₃) δ 8.14-8.08; (m, 2H), 7.63-7.56; (m, 1H), 7.49; (t, J=7.8 Hz, 2H), 7.44-7.37; (m, 2H), 7.37-7.29; (m, 3H), 6.29; (t, J=9.2 Hz, 1H), 5.65; (d, J=7.0 Hz, 1H), 5.46; (s, 1H), 5.39; (d, J=9.8 Hz, 1H), 5.25; (d, J=2.6 Hz, 1H), 4.99; (dd, J=9.8, 2.1 Hz, 1H), 4.82; (s, 1H), 4.34-4.24; (m, 2H), 4.22-4.03; (m, 5H), 3.90; (dd, J=10.8, 6.4 Hz, 1H), 3.85; (d, J=6.9 Hz, 1H), 3.68; (td, J=8.5, 6.4 Hz, 1H), 3.44; (s, 3H), 3.30; (s, 3H), 2.70; (ddd, J=14.2, 9.8, 6.4 Hz, 1H), 2.44; (s, 3H), 2.36-2.17; (m, 2H), 2.00; (d, J=1.5 Hz, 3H), 1.79; (ddd, J=13.4, 10.9, 2.2 Hz, 1H), 1.72; (s, 3H), 1.64-1.53; (m, 4H), 1.35; (s, 9H), 1.33-1.23; (m, 12H), 1.22; (s, 3H), 1.20; (s, 3H), 0.88; (td, J=7.1, 1.6 Hz, 6H). ¹³C NMR (126 MHz, CDCl₃) δ 205.09, 169.86, 168.13, 168.11, 167.18, 154.32, 139.54, 135.23, 133.73, 130.31, 129.40, 129.09, 128.79, 128.43, 126.57, 113.69, 84.31, 82.67, 81.76, 80.83, 80.63, 79.02, 76.62, 74.89, 73.19, 72.52, 68.92, 66.64, 57.30, 57.21, 56.97, 47.50, 43.51, 37.40, 36.92, 35.10, 32.21, 32.18, 32.16, 32.14, 28.26, 26.81, 23.74, 23.42, 22.97, 22.72, 21.16, 14.58, 14.34, 14.17, 10.52. HRMS (ESI): m/z [M+Na]⁺ calcd. for C₆₀H₈₄NO₁₈, 1128.5610; found, 1128.5492.

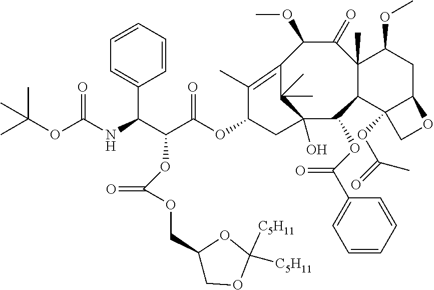

NCC-SA-09-(2aR,4S,4aS,6R,9S,11S,12S,12aR,12bS)-12b-acetoxy-9-(((2R,3S)-3-((tert-butoxycarbonyl)amino)-2-(((((S)-2,2- dipentyl-1,3-dioxolan-4-yl)methoxy)carbonyl)oxy)-3-phenylpropanoyl)oxy)-11-hydroxy-4,6-dimethoxy-4a,8,13,13 -tetramethyl-5-oxo-2a,3,4,4a,5,6,9,10,11,12,12a,12b-dodecahydro-1H-7,11-methanocyclodeca[3 ,4]benzo[1,2-b]oxet-12-yl benzoate: ¹H NMR (500 MHz, CDCl₃) δ 8.11; (d, J=7.3 Hz, 2H), 7.63-7.56; (m, 1H), 7.49; (t, J=7.8 Hz, 2H), 7.41; (t, J=7.5 Hz, 2H), 7.37-7.29; (m, 3H), 6.29; (t, J=9.3 Hz, 1H), 5.65; (d, J=7.0 Hz, 1H), 5.46; (brs, 1H), 5.39; (d, J=10.0 Hz, 1H), 5.25; (dd, J=6.6, 2.5 Hz, 1H), 4.99; (dd, J=9.9, 2.2 Hz, 1H), 4.82; (s, 1H), 4.34-4.25; (m, 2H), 4.22-4.09; (m, 3H), 4.11-4.03; (m, 1H), 3.90; (dd, J=10.7, 6.4 Hz, 1H), 3.85; (d, J=7.0 Hz, 1H), 3.66; (dd, J=8.5, 6.5 Hz, 1H), 3.44; (s, 3H), 3.30; (s, 3H), 2.70; (ddd, J=14.1, 9.8, 6.4 Hz, 1H), 2.43; (s, 3H), 2.36-2.17; (m, 2H), 2.00; (s, 3H), 1.79; (ddd, J=13.8, 10.9, 2.3 Hz, 1H), 1.71; (s, 3H), 1.67-1.55; (m, 4H), 1.34; (s, 9H), 1.34-1.23; (m, 12H), 1.22; (s, 3H), 1.20; (s, 3H), 0.88; (td, J=7.1, 2.2 Hz, 6H). ¹³C NMR (126 MHz, CDCl₃) δ 205.10, 169.88, 168.14, 167.18, 154.33, 139.52, 135.24, 133.73, 130.31, 129.40, 129.11, 128.79, 128.43, 126.56, 113.75, 84.32, 82.68, 81.77, 80.83, 80.64, 79.02, 76.63, 74.89, 73.32, 72.53, 69.17, 66.82, 57.31, 57.21, 56.98, 47.50, 43.51, 37.43, 36.96, 35.10, 32.21, 32.15, 28.26, 26.81, 23.74, 23.41, 22.97, 22.72, 21.17, 14.59, 14.19, 10.52. HRMS (ESI): m/z [M+H]⁺ calcd. for C₆₀H₈₄NO₁₈, 1106.5610; found, 1106.5692.

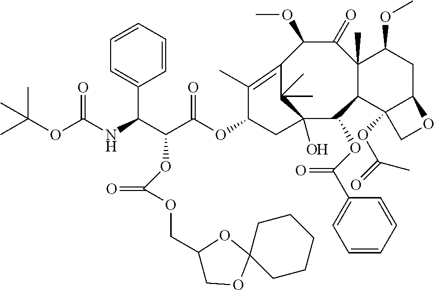

NCC-SA-10-(2aR,4S,4aS,6R,9S,11S,12S,12aR,12bS)-9-(((2R,3S)-2-((((1,4-dioxaspiro[4.5]decan-2-yl)methoxy)carbonyl)oxy)-3-((tert-butoxycarbonyl)amino)-3-phenylpropanoyl)oxy)-12b-acetoxy-11-hydroxy-4,6-dimethoxy-4a,8,13,13-tetramethyl-5-oxo-2a,3,4,4a,5,6,9,10,11,12,12a,12b- dodecahydro-1H-[7,11]methanocyclodeca[3,4]benzo[1,2-b]oxet-12-yl benzoate: ¹H NMR (500 MHz, Chloroform-d) δ 8.13-8.08; (m, 2H), 7.63-7.56; (m, 1H), 7.49; (t, J=7.8 Hz, 2H), 7.44-7.37; (m, 2H), 7.37-7.29; (m, 3H), 6.28; (s, 1H), 5.65; (d, J=7.0 Hz, 1H), 5.47-5.39; (m, 2H), 5.25; (dd, J=7.8, 2.5 Hz, 1H), 4.99; (dd, J=9.1, 2.0 Hz, 1H), 4.82; (s, 1H), 4.34-4.25; (m, 2H), 4.21-4.09; (m, 3H), 4.05; (ddd, J=8.5, 6.4, 2.1 Hz, 1H), 3.90; (dd, J=10.7, 6.4 Hz, 1H), 3.85; (d, J=6.9 Hz, 1H), 3.75; (ddd, J=16.0, 8.6, 5.6 Hz, 1H), 3.44; (d, J=1.1 Hz, 3H), 3.30; (s, 3H), 2.70; (ddd, J=14.1, 9.7, 6.3 Hz, 1H), 2.44; (d, J=3.1 Hz, 3H), 2.00; (s, 3H), 1.79; (ddd, J=13.9, 11.0, 2.2 Hz, 1H), 1.71; (s, 3H), 1.65-1.53; (m, 9H), 1.45-1.39; (m, 1H), 1.34; (s, 9H), 1.22; (s, 3H), 1.20; (s, 3H). ¹³C NMR (126 MHz, CDCl₃) δ 205.09, 169.88, 168.16, 168.15, 167.18, 154.30, 135.22, 133.73, 130.31, 129.40, 129.10, 129.08, 128.79, 128.42, 126.57, 110.84, 110.74, 84.31, 82.67, 81.75, 80.83, 80.63, 79.02, 76.62, 74.89, 72.84, 72.75, 72.52, 69.19, 68.77, 66.07, 65.86, 57.30, 57.29, 57.21, 56.98, 47.50, 43.50, 36.50, 36.39, 35.10, 34.94, 34.88, 32.18, 28.27, 26.82, 25.17, 24.10, 24.06, 23.88, 22.96, 22.60, 21.17, 14.59, 14.07, 10.52. HRMS (ESI): m/z [M+Na]⁺ calcd. for C₅₅H₇₁NO₁₉, 1056.4671; found, 1056.4549.

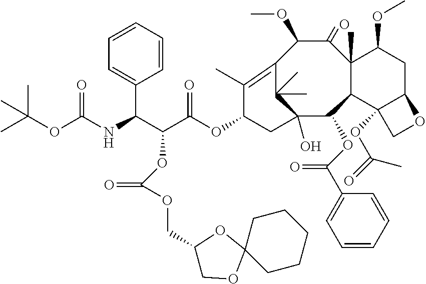

NCC-SA-12-(2aR,4S,4aS,6R,9S,11S,12S,12aR,12bS)-9-(((2R,3S)-2-(((((R)-1,4-dioxaspiro[4.5]decan-2- yl)methoxy)carbonyl)oxy)-3-((tert-butoxycarbonyl)amino)-3-phenylpropanoyl)oxy)-12b-acetoxy-11-hydroxy-4,6-dimethoxy-4a,8,13,13-tetramethyl-5-oxo-2a,3,4,4a,5,6,9,10,11,12,12a,12b-dodecahydro-1H-[7,11]methanocyclodeca[3,4]benzo[1,2-b]oxet-12-yl benzoate: ¹H NMR (500 MHz, CDCl₃) δ 8.13-8.08; (m, 2H), 7.63-7.56; (m, 1H), 7.49; (t, J=7.8 Hz, 2H), 7.44-7.36; (m, 2H), 7.36-7.29; (m, 3H), 6.29; (t, J=9.0 Hz, 1H), 5.65; (d, J=7.0 Hz, 1H), 5.44; (d, J=12.3 Hz, 2H), 5.25; (dd, J=7.8, 2.4 Hz, 1H), 5.02-4.96; (m, 1H), 4.82; (s, 1H), 4.29; (dt, J=10.8, 7.1 Hz, 2H), 4.21-4.10; (m, 3H), 4.05; (ddd, J=8.6, 6.4, 2.1 Hz, 1H), 3.89; (dd, J=10.7, 6.4 Hz, 1H), 3.85; (d, J=7.0 Hz, 1H), 3.74; (ddd, J=15.9, 8.7, 5.6 Hz, 1H), 3.44; (d, J=1.1 Hz, 3H), 3.30; (s, 3H), 2.70; (ddd, J=14.1, 9.8, 6.4 Hz, 1H), 2.43; (d, J=3.3 Hz, 3H), 2.37-2.16; (m, 2H), 2.00; (s, 3H), 1.79; (ddd, J=13.9, 11.0, 2.2 Hz, 1H), 1.71; (s, 3H), 1.65-1.53; (m, 9H), 1.45-1.39; (m, 1H), 1.34; (s, 9H), 1.22; (s, 3H), 1.20; (s, 3H). ¹³C NMR (126 MHz, CDCl₃) δ 205.09, 169.87, 168.16, 168.14, 167.17, 154.29, 139.56, 135.20, 133.72, 130.30, 129.40, 129.09, 128.79, 128.42, 126.57, 110.79, 110.74, 84.31, 82.66, 81.75, 80.82, 80.62, 79.01, 76.62, 74.89, 72.75, 72.51, 68.76, 65.85, 57.29, 57.21, 56.97, 47.49, 43.50, 36.39, 34.93, 34.88, 32.17, 28.26, 26.81, 25.17, 24.05, 23.87, 22.95, 21.16, 14.59, 10.52. HRMS (ESI): m/z [M+Na]⁺ calcd. for C₅₅H₇₁NO₁₈, 1056.4671; found, 1056.4555.

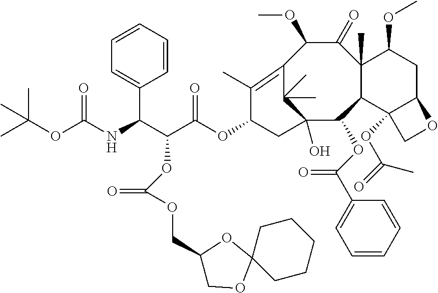

NCC-SA-11-(2aR,4S,4aS,6R,9S,11S,12S,12aR,12bS)-9-(((2R,3S)-2-(((((S)-1,4-dioxaspiro[4.5]decan-2- yl)methoxy)carbonyl)oxy)-3-((tert-butoxycarbonyl)amino)-3-phenylpropanoyl)oxy)-12b-acetoxy-11-hydroxy-4,6-dimethoxy-4a,8,13,13-tetramethyl-5-oxo-2a,3 ,4,4a,5,6,9,10,11,12,12a,12b-dodecahydro-1H-[7,11]methanocyclodeca[3,4]benzo[1,2-b]oxet-12-yl benzoate: ¹H NMR (500 MHz, CDCl₃) δ 8.13-8.08; (m, 2H), 7.63-7.56; (m, 1H), 7.49; (t, J=7.7 Hz, 2H), 7.44-7.37; (m, 2H), 7.36-7.30; (m, 3H), 6.28; (t, J=9.0 Hz, 1H), 5.65; (d, J=7.0 Hz, 1H), 5.46; (s, 1H), 5.40; (d, J=10.3 Hz, 1H), 5.25; (dd, J=8.0, 2.5 Hz, 1H), 4.99; (dd, J=9.7, 2.1 Hz, 1H), 4.82; (s, 1H), 4.34-4.26; (m, 2H), 4.21-4.14; (m, 2H), 4.17-4.09; (m, 1H), 4.05; (ddd, J=8.5, 6.4, 2.2 Hz, 1H), 3.89; (dd, J=10.7, 6.4 Hz, 1H), 3.85; (d, J=7.0 Hz, 1H), 3.74; (td, J=8.7, 8.1, 5.6 Hz, 1H), 3.44; (s, 3H), 3.30; (s, 3H), 2.70; (ddd, J=14.1, 9.8, 6.4 Hz, 1H), 2.44; (d, J=3.1 Hz, 3H), 2.37-2.16; (m, 2H), 2.00; (s, 3H), 1.79; (ddd, J=13.9, 11.0, 2.2 Hz, 1H), 1.71; (s, 3H), 1.64-1.53; (m, 9H), 1.45-1.39; (m, 1H), 1.34; (s, 9H), 1.22; (s, 3H), 1.20; (s, 3H). ¹³C NMR (126 MHz, CDCl₃) δ 205.09, 169.88, 168.14, 167.17, 154.30, 139.53, 135.22, 133.73, 130.31, 129.40, 129.09, 128.79, 128.43, 126.57, 110.79, 84.31, 82.67, 81.75, 80.82, 80.63, 79.02, 76.62, 74.88, 72.84, 72.52, 69.19, 66.06, 57.30, 57.21, 56.97, 47.49, 43.50, 36.50, 35.10, 34.88, 32.17, 28.26, 26.81, 25.17, 24.10, 23.87, 22.95, 21.16, 14.59, 10.52. HRMS (ESI): m/z [M+Na]⁺ calcd. for C₅₅H₇₁NO₁₈, 1056.4671; found, 1056.4548.

### Chromatographic Isolation of Chiral Compounds (Diastereomerically Pure Compounds)

In one method, when a mixture of diastereomeric compounds is obtained in the synthesis, purification or isolation of a single diastereomer may also be performed using a silica-based ion-exchange, such as using chemically-bonded sulfonic acid groups, with silver ions. Accordingly, the preparation of the silver impregnated columns involves the use of a standard pre-packed column with an appropriate stationary phase, such as Nucleosil™ SSA, and introducing the silver ions via a Rheodyne™ injector while pumping water through the column. The aqueous phase is then replaced with organic solvents. Typically, using this method, about 50 mg to 80 mg of silver ions are bound to the stationary phase. Silver ion columns may also be obtained from commercial sources, such as Chromspher Lipids™, Varian-Chrompack International, Middelburg, Netherlands. See Morris, L. J. Separation of lipids by silver ion chromatography. J. Lipid Res., 7, 717-732 (1966).

### Formulation of Compounds

The following Table is a representative composition of a prepared formulation using a representative compound prepared herein:

| Description: | Vehicle with Miglyol |
|---|---|
| | 812N Formulation |

| | | | |
|---|---|---|---|
| Prep | 100 mL | Prep Type: | DCM Film |
| Volume: | | Target API Prep | 0.00 mg/mL |
| Solvent: | DCM | Concentration: | |
| Buffer: | Acetate, pH 5.5, + NS | Processing Temperature: | 60° C. |

| | | Amount | Actual | | |
|---|---|---|---|---|---|
| | | Weighed | mg/mL in | Actual | |
| Ingredient | | (g) | Emulsion | % w/w | % Target |
| 1 | Egg Yolk (Lipoid E 80); 80% | 4.2578 | 42.58 | 63.59% | 100.18% |
| | Phosphatidylcholine (PC80) | | | | |
| 2 | Dimyristoylphosphocholine (DMPC) | 1.1400 | 11.40 | 17.03% | 101.79% |
| 3 | Poloxamer P188 (P188) | 0.3063 | 3.06 | 4.57% | 102.10% |
| 4 | Cholesterol Oleate (CE) | 0.1506 | 1.51 | 2.25% | 100.40% |
| 5 | Cholesterol (Free; FC) | 0.0709 | 0.71 | 1.06% | 101.29% |
| | ART207 | 0.0000 | 0.00 | 0.00% | 0.00% |
| 6 | Ubiquinol 10 (U) | 0.0108 | 0.11 | 0.16% | 108.00% |
| 7 | Mixed Tocopherols (VitE) | 0.0118 | 0.12 | 0.18% | 118.00% |
| 8 | Miglyol 812N (M812N) | 0.7475 | 7.48 | 11.16% | 101.01% |

| | | | | |
|---|---|---|---|---|
| Total: | 6.6957 | 6.6957 | 66.96 | 100.00% |

The compound (the API) may be selected and added to the composition as represented in the above table, to obtain a final concentration of the formulated product, to provide, for example, 0.500 grams of the compound, such as ART 207, to formulate a formulation product at about 5 mg/ml. The Miglyol 812N (a C8/C10 triglyceride (MCT oil)) may be substituted with other triglyceride esters or other medium-chain triglycerides as disclosed herein.

### The Following General Procedure was Used to Prepare the Composition

Weigh the first five (5) ingredients into individual weigh boats and transfer the contents from directly into a 400-mL beaker. Weigh the next three (3) ingredients into a single Scintillation vial. Add approximately 5 mL DCM to the Scintillations vial cap, and mix by inversion. Transfer the contents from the vial to the beaker. Rinse the vial 2 times with approximately 5 mL of DCM and add each wash to the beaker (total DCM volume ˜15 mL; total solution volume ˜18 mL). Cover the beaker with a watch glass and allow the DCM to reflux in the 45° C. oven until all the ingredients have dissolved; swirl every few minutes. Approximate time: 15 minutes.

Remove the watch glass, place the beaker in the water bath at 60° C. and direct a gentle stream of nitrogen across the surface of the DCM mixture to create a gentle turbulence. Continue to evaporate the DCM using gentle agitation until the mixture forms a viscous film.

Place the beaker into a 45° C. vacuum oven at full vacuum for NLT one (1) hour; decrease the vacuum if the mixture swells above 200 mL. Place the beaker into the 60° C. water bath and then add ˜94 mL of pre-warmed (60° C.) 10 mM sodium acetate pH 5.5 buffer (see ELN 2020Dec07_R_D_dle_039) and swirl to yield a homogeneous milky solution.

Blend with Oster hand blender, on low speed, using one (1) to two (2) second pulses; continue pulsing for three (3) minutes (NLT two (2) minutes). Measure the particle size. Continue pulsing with the hand blender for two (2) minutes using ten (10) second pulses. Measure the particle size.

Transfer the resultant solution to the Microfluidizer inlet reservoir and pass the mixture through the Microfluidizer (60° C., 25k psi) for a minimum of 10 passes. Measure particle size after each second pass (10a through 10e minimum). Continue with additional passes as necessary while monitoring particle size to assess filterability.

Collect the product from Microfluidizer holdup volume using 30 mL of buffer and exactly four (4) pump strokes to yield ˜100 mL of processed material. Transfer the solution to a 100 mL graduated cylinder and verify the final volume; take samples for particle size and potency. Filter the resulting solution through a single Nalgene 0.2 μm aPES filter until the filter becomes clogged. Use an additional filter or filters as needed. Measure the particle size of each filtrate. Measure potency of each filtrate. Add 100 mL of IPA to the Microfluidizer feed hooper and dispose of the remainder of the buffer flush and IPA (MF Wash 1). Note: This wash contains both IPA and residual buffer from within the mixing chamber. Rinse the Microfluidizer with another 100 mL of IPA (MF Wash 2).

### NCI-60 Cell One-Dose and Five Dose Screening Data

The human tumor cell lines of the cancer screening panel are grown in RPMI 1640 medium containing 5% fetal bovine serum and 2 mM L-glutamine. For a typical screening experiment, cells are inoculated into 96-well microtiter plates in 100 μL at plating densities ranging from 5,000 to 40,000 cells/well depending on the doubling time of individual cell lines. After cell inoculation, the microtiter plates are incubated at 37° C., 5% CO2, 95% air and 100% relative humidity for 24 h prior to addition of experimental drugs.

After 24 hours (hrs), two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 m/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37 ° C., 5% CO2, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

[(Ti−Tz)/(C−Tz)]×100 for concentrations for which Ti>/=Tz

[(Ti−Tz)/Tz]×100 for concentrations for which Ti<Tz.

Three dose-response parameters are calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti−Tz. The LC50 (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

### Cell Culture

Breast (MDA-MB-231 and MDA-MB-453), pancreatic (MIA-paca-2), lung (A549 and NIH-1975), prostate (DU145) and ovarian (SKOV-3) cancer cell lines were obtained from ATCC (Manassas, Va.). Cancer cell lines were maintained in culture in their respective culture media (DMEM or RPMI, per ATCC recommendations), supplemented with 10% Fetal Bovine Serum (Atlanta Biological, Ga.), and 1% penicillin/streptomycin (Invitrogen-Gibco, Carlsbad, Calif.) at 37° C. in a humidified chamber with 5% CO2.

### Cell Survival Assay

Cancer cells were seeded at 3,000 cells/well in 384-well plate format. After 24 hrs, cells were treated with saline (0.9% NaC1), Abraxane®, vehicle and ART-207 at doses reported in Results section for 48 and 72 hrs. Inhibition was determined by adding Cell Counting Kit-8 (CCK8, APExBio, Houton, Tex.). After 2 hrs of incubation at 3 ° C., optical density was measured at 450 nm using a SpectraMax M3 spectrophotometer (Molecular Devices, San Jose, Calif.).

### Tumor Xenograft Inhibition Assay

6-8 weeks old NOD-SCID-NOD. Cg-Prkdc^{scid} I12re^{tm1/Wj1}/SzJ mice were obtained from Jackson Laboratories (Bar Harbor, Me.) and acclimated in the laboratories for six days prior to experimentation. Mice were maintained in the Laboratory of Animals Facility of the Mississippi Medical Center (Jackson, Miss.). All animals were housed in microisolator cages, up to five per cage, in a 12-hr light/dark cycle. The animals received filtered Jackson municipal water and sterilized rodent diet (Teklad LM-485 Mouse/Rat Diet 7912, ENVIGO) ad libitum. Cages were changed twice weekly. The animals were observed daily and clinical signs were noted. All experimental procedures were approved by the Institutional Animal Care and Use Committee of the University of Mississippi Medical Center (UMMC). Animal laboratories of UMMC are AAALAC accredited. 2×10⁶ SKOV-3 ovarian cancer cells in 50 μL of PBS were injected with 50 μL of Matrigel (Corning, location) subcutaneously on the right flank of mice. Tumor's size was measured using a caliper and tumor volumes were calculated using the formula: Tumor volume=length×width/2, where length represents the largest tumor diameter and width represents the perpendicular tumor diameter. Mice were monitored daily until tumors reached 150-200 mm³ size. A first round of treatment was injected during 5 consecutive days and a second round of 5 days treatment was administered after 2 weeks. At the end of experiment mice were euthanized and tumors were collected and weighed.

### Cytotoxicity of Specific Compounds

### MTS Proliferation Assay Using SK-N-AS Cells

Day 1: SK-N-AS cells are plated in appropriate growth medium at 5×10³ per well in 100 μL in 96 well tissue culture plates, Falcon, one plate for each compound to be tested. Column 1 was blank; it contained medium, but no cells. The plates are incubated overnight at 37° C. in 5% CO2 to allow attachment.

Day 2: Drug diluted in culture media is added to the cells at a concentration of 0.005 nM to 10 μM, in quadruplicate. After 48-72 hrs of drug exposure, the MTS agent is added to all wells and incubated 1-6 hrs (37° C., 5% CO₂), depending on cell type, as per CellTiter 96® AQueous Non-Radioactive Cell Proliferation Assay (MTS), Promega. Plates are processed using a Bio-Tek Synergy HT Multi-detection microtiter plate reader at 490 nanometer wavelength and data were processed with KC4V.3 software. Data plots of drug concentration vs. absorbance are plotted and the concentration resulting in 50% inhibition (IC50) is extrapolated for each of the tested compounds.

### MTT Proliferation Assay Using Paired MDR+ and MDR− Cell Lines

A second evaluation of the cytotoxicity of the acid labile, lipophilic molecular conjugates was undertaken. The purpose of these experiments was to compare the toxicity of the conjugates in multidrug resistant cells and their parental susceptible lines to test the hypothesis that a subset of these compounds would exhibit a similar level of toxicity in the drug resistant lines as that observed in the parent susceptible cell line.

MTT-based cytotoxicity assays were performed using human cancer cell lines and paired sublines exhibiting multidrug resistance. These lines included a uterine sarcoma line, MES-SA, and its doxorubicin-resistant subline, MES-SA/Dx5. See W. G. Harker et al. Development and characterization of a human sarcoma cell line, MES-SA, sensitive to multiple drugs. Cancer Research 43: 4943-4950 (1983); W. G. Harker et al. Multidrug (pleiotropic) resistance in doxorubicin-selected variants of the human sarcoma cell line MES-SA. Cancer Research 45: 4091 4096 (1985).

The stability of the acid labile, lipophilic molecular conjugates to hydrolysis in plasma is evaluated to determine their potential to release the active cancer chemotherapeutic agents into systemic circulation and thereby cause general off target toxicity (“side effects”). The conjugates are incubated with plasma of mouse, rat and human origin.

HPLC grade Methanol from Fisher (Fair lawn, N.J., USA). Part No: A452-4 (074833). HPLC grade Water from Fisher (Fair lawn, N.J., USA). Part No: W5-4 (073352). Drug-free mouse, rat and human plasmas were purchased from Innovative Research Inc. (Southfield, Mich., USA). Liposyn® I.V. Fat Emulsion from Hospira, Inc. (Lake Forest, Ill.).

### Preparation of Plasma Incubations

Each compound is prepared in triplicate in mouse, rat and human plasma individually at 10 μg/ml concentration and vortexed for 1 minute and placed in a water bath at 37° C. at a shake rate of 75 per minute. Samples are drawn at time points of 0, 15, 30, 45, 60, 75, 90, 105, 120, 135, 150, 165, 180, 210, 240, 300, 360 and 480 minutes. Analytical Method for the compounds prepared herein, as analyzed in plasma:

Chromatographic separation of the compounds is performed on a Waters Acquity UPLC™ using a BEH C18 column (1.7 μm, 2.1×50 mm). The mobile phase consisted of Methanol: 0.1% Formic acid (80:20). The flow rate is 0.3 ml/min; the sample injection volume was 5 μL, resulting in a 3 minute run time.

The MS instrumentation consisted of a Waters Micromass Quattro Micro™ triple-quadrapole system (Manchester, UK). The MS system is controlled by a 4.0 version of MassLynx software. Ionization is performed in the positive electrospray ionization mode. MS/MS conditions are the following: capillary voltage 3.02 kV; cone voltage 50 v; extractor voltage 5 v; and RF lens voltage 0.5 v. The source and desolvation temperatures are 100° C. and 400° C. respectively, and the desolvation and cone gas flow are 400 and 30 L/hr, respectively.

The selected mass-to-charge (m/z) ratio transitions of the compounds used in the selected ion monitoring (SIM) are as noted. The dwell time was set at 200 msec. MS conditions are optimized using direct infusion of standard solutions prepared in methanol and delivered by a syringe pump at a flow rate of 20 μL/min.

### Plasma Sample Preparation

Samples of 100 μL are collected at time points of 0, 15, 30, 45, 60, 75, 90, 105, 120, 135, 150, 165, 180, 210, 240, 300, 360 and 480 minutes respectively and the reaction was terminated with methanol. In a separate set of experiments the acid labile, lipophilic molecular conjugates are dissolved in a small amount of ethanol and diluted into a lipid emulsion (Liposyn®) and added to mouse and human plasma before incubation and the hydrolysis of the conjugates is similarly measured. Collected plasma samples of 100 μL containing a selected compound are placed in separate Eppendorf micro centrifuge tubes for processing. Methanol (200 μL) is added to extract the drug using the protein precipitation technique. The micro tubes are then vortex mixed for 10 minutes and centrifuged for 15 minutes at a speed of 10,000 rpm (Eppendorf 5415C centrifuge). The supernatant is collected and filtered using a 0.45 μm filter (Waters 13mm GHP 0.45 μm) before analysis.

UPLC/MS/MS analysis of blank mouse, rat and human plasma samples shows no endogenous peak interference with the quantification of the above compounds.

The weighted linear least-squares (1/×) regression is used as the mathematical model. The coefficient (r) for the compounds ranged from 0.9925 to 0.9999. The calibration range is selected according to the concentrations anticipated in the samples to be determined. The final calibration range was 10-12,500 ng/mL with a lower limit of quantification of 10 ng/mL. The repeatability and reproducibility bias (%) is within the acceptance limits of ±20% at low concentration and ±15% at other concentration levels with RSD's of less than 5% at all concentrations evaluated.

The mean recoveries of the method are in the range of 86.22-99.83% at three different concentrations of the test compounds from plasma. These results suggested that there was no relevant difference in extraction recovery at different concentration levels.

### Incubations of the Prepared Compounds

A 0.2 ml aliquot from 210.6 μg/ml stock solution of a selected compound prepared herein is spiked into 3.8 ml of human plasma preincubated for 15 min (37° C.) and incubated in a reciprocating water bath at 37° C. Samples are drawn at 0, 0.5, 1, 2, 3, 4, 6, 8, 10, 12 and 24 hrs. Analytical Method for the prepared compounds (Liquid Chromatography-Tandem Mass Spectrometry):

Chromatographic separation was carried out using an ACQUITY UPLC liquid chromatograph (Waters Corporation, Milford, Mass., USA) consisting of a binary pump, autosampler, degasser and column oven. A mobile phase of methanol-acetonitrile (50: 50, v/v) is pumped at a flow-rate of 0.4 ml/min through an ACQUITY UPLC BEH C18 column (1.7 μm, 2.1×50 mm i.d., Waters Corporation) maintained at 25° C. 10 μl of sample is injected and the run time was 3.0 min. The LC elute is connected directly to an ESCi triple-quadrapole mass spectrometer equipped with an electrospray ionization (ESI) ion source. The quadrapoles are operated in the positive ion mode. The multiple reaction monitoring (MRM) mode is used for quantification using MassLynx version 4.1 software. Mass transitions of selected m/z are optimized for the selected compound with dwell time of 0.5 s. Nitrogen is used as nebulizing gas (30 l/h) and desolvation gas (300 l/h) with a desolvation temperature at 250° C., and argon as collision gas. The capillary voltage is set at 3.5 kV, and cone voltage at 90 V; and the source temperature is set at 100° C.

### Plasma Sample Preparation

At the different time periods (0, 0.5, 1, 2, 3, 4, 6, 8, 10, 12 and 24 h), 200 μl aliquot of samples are taken and immediately added to 1.3 ml of cold TBME and subsequently 20 μl of internal standard stock solution (80.7 μg/ml in methanol) is added. Each tube is vortex mixed for approximately 2 min and then centrifuged at 13000 rpm for 10 min. 1.0 ml of resultant supernatant is transferred to another tube and dried under a stream of nitrogen gas at 35 ° C. Each dried residue is reconstituted with 200 μl of methanol and vortex mixed for 0.5 min. After centrifugation at 13000 rpm for 10 min, the supernatants are transferred to HPLC autosampler vials, and 10 μl aliquot of each sample is injected into LC-MS-MS.

Samples are collected at various times and the per cent remaining of the acid labile, lipophilic molecular conjugate of the compound is determined along with the per cent of the chemotherapeutic agent released from the hydrolysis of the conjugate.

A panel of the lipophilic, acid-labile paclitaxel conjugates disclosed herein were screened at the National Cancer Institute's NCI-60 Human Tumor Cell Lines Screen program (the methodology for NCI-60 cell line screening is described at htps://dtp.cancer.gov/discorvery_development/nci-60/) to probe their cytotoxicity and compare with the parent, paclitaxel. The panel was organized into nine subpanels representing diverse histologies: leukemia, melanoma, lung, colon, kidney, ovary, breast, prostate, and central nervous system. The screening was a two-stage process, beginning with evaluating all compounds against the 60 cell lines at a single dose of 10 μM in EtOH. All samples were solubilized in ethanol instead of the standard solvent, DMSO. The results of the reference drug paclitaxel (NSC125793) were retrieved from publicly available sources, http://dtp.nci.nih.gov. The in vitro results of single-dose results for the conjugates were tabulated as radar charts to comprehend the cytotoxicity of multiple analogs against multiple tested cell lines. Significant cytotoxicity of all conjugates against ovarian cancer cell lines, including paclitaxel-resistant cell line, SK-OV-3, were noted. The conjugates produced a similar sensitivity and resistance profile to that of paclitaxel to NCI-60 cells. The conjugates exhibited significant growth inhibition, and were evaluated against the NCI-60 cell lines at five concentration levels to a final concentration of 0.1 nM (Supporting information, S6) to establish GI50 values; which showed superior activity in view of the corresponding non-conjugated compounds.

The capacity to reduce the survival of cancer cells, SK-OV-3 cells were incubated with the formulated conjugates was compared with Abraxane®, an FDA-approved paclitaxel formulation, by varying the exposure time and dosage. Even after 72 h of exposure, the EC50 of the conjugate with pseudo-LDL nanoparticle formulations was at least 3× more potent than Abraxane® independent of concentrations tested.

These and other objects of the present invention will become more readily appreciated and understood from a consideration of the following detailed description of the exemplary embodiments of the present invention when taken together with the accompanying drawings. The entire disclosures of all documents cited throughout this application are incorporated herein by reference.

What is claimed is:

1. An acid labile lipophilic molecular conjugate (ALLMC) of the formulae: 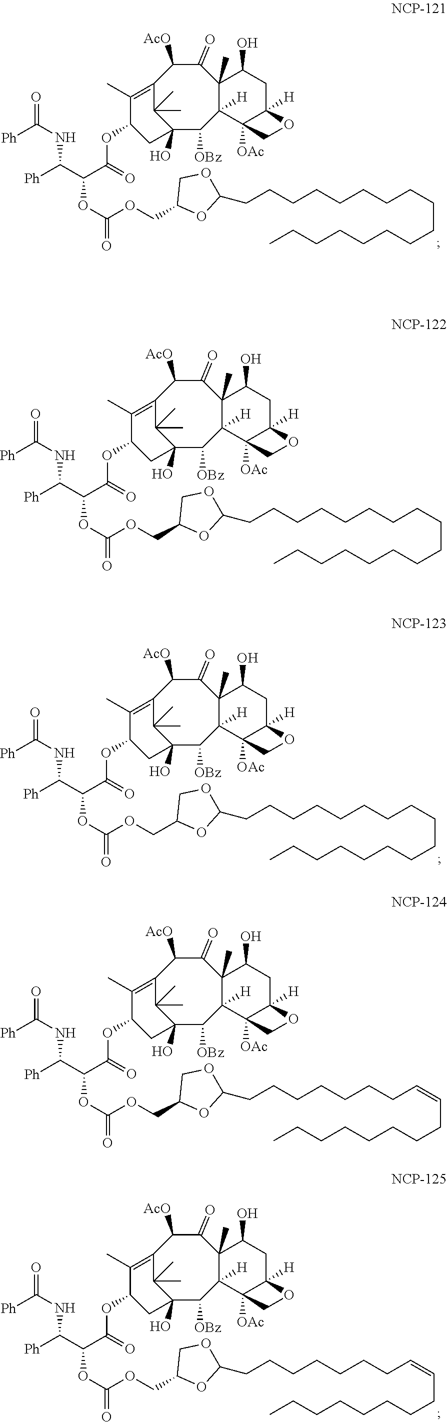 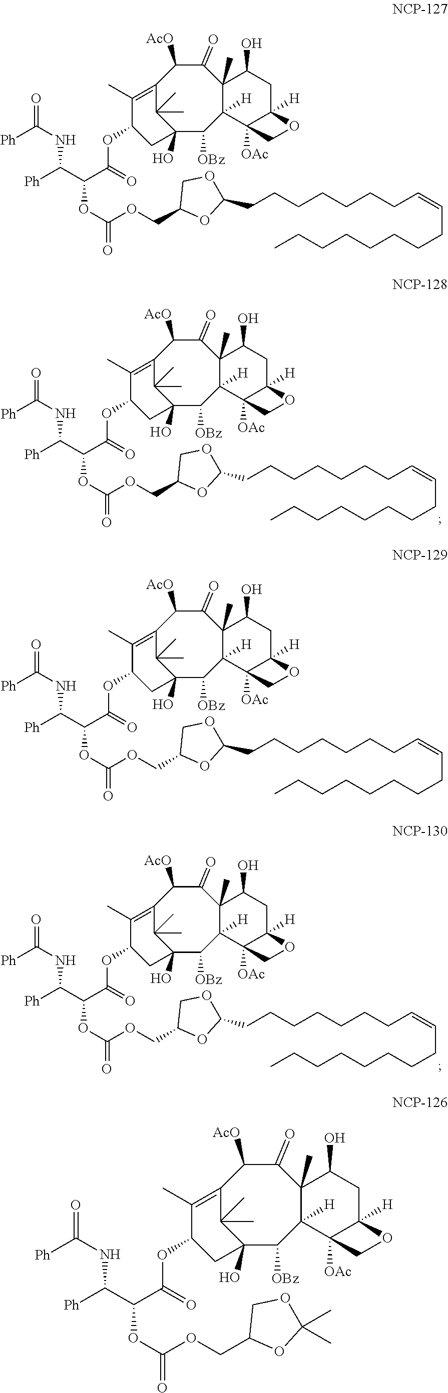 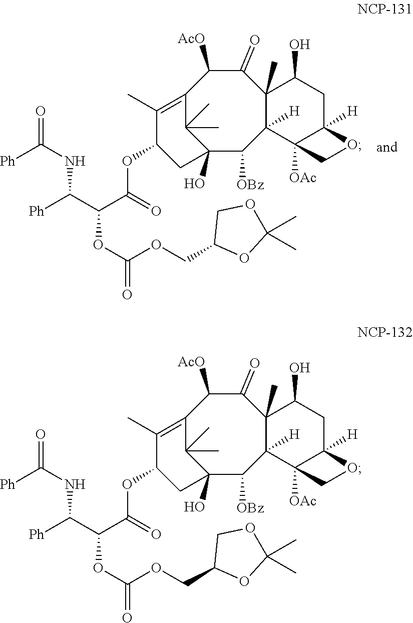
and their isolated diastereoisomers or mixtures thereof; or
a pharmaceutically acceptable salt thereof.

2. An acid labile lipophilic molecular conjugate (ALLMC) of the formulae:

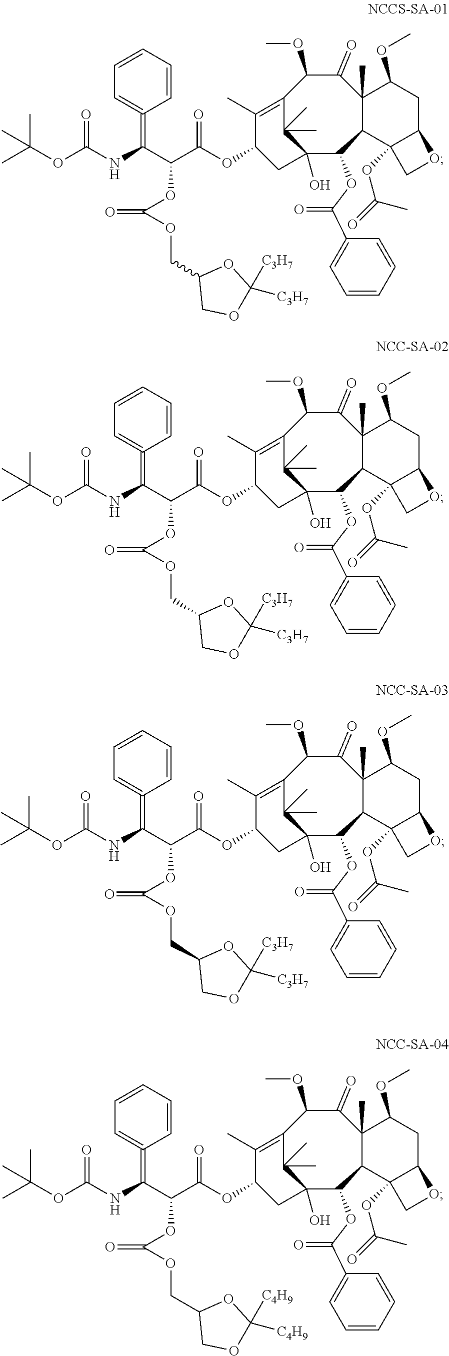

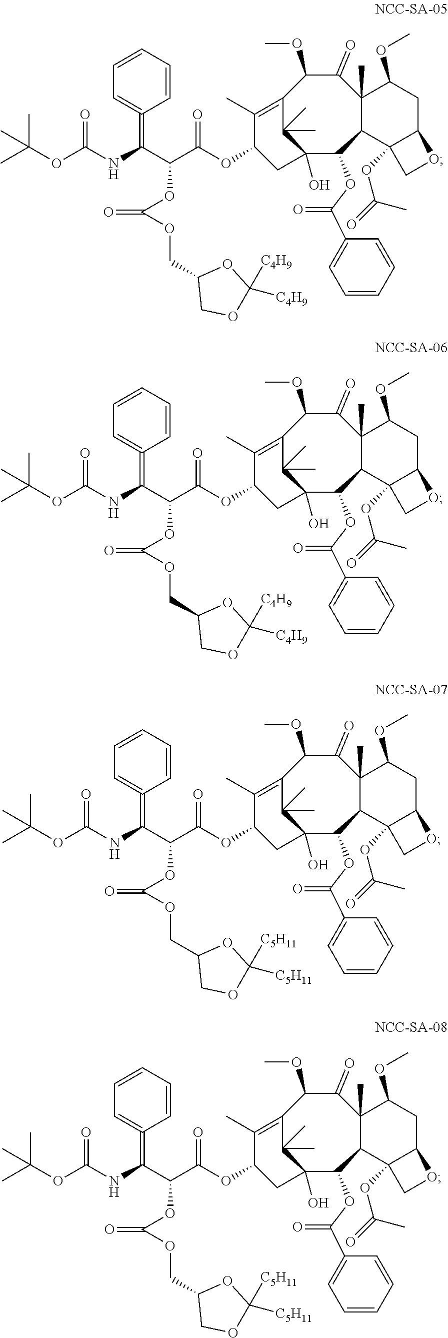

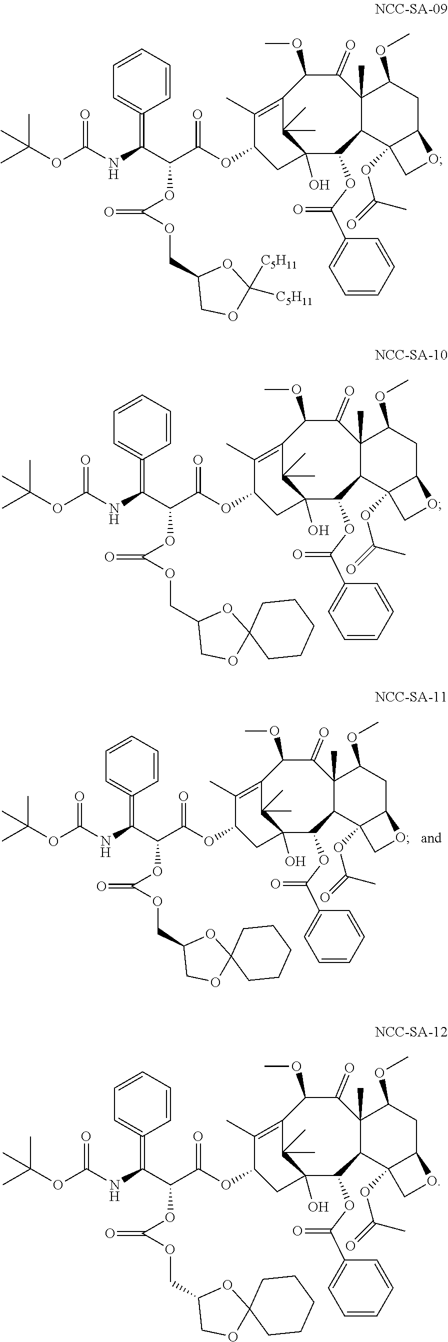

3. The acid labile lipophilic molecular conjugate of claim 1 that is (2aR,4S,4aS,6R,9S,11S,12S,12aR,12bS)-9-(((2R,3S)-3-benzamido-2-((((2,2-dimethyl-1,3-dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2- b]oxete-6,12b(2aH)-diyl diacetate (NCP-126) and of the formula:

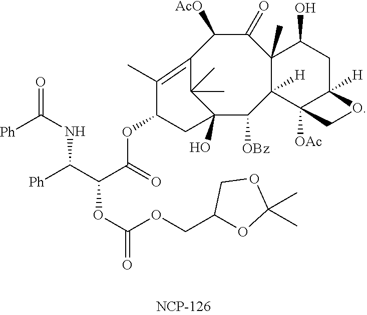

4. The acid labile lipophilic molecular conjugate of claim 1 that is (2aR,4S ,4aS ,6R,9S,11S,12S,12aR,12bS)-9-(((2R,3S)-3-benzamido-2-(((((S)-2,2-dimethyl-1,3 -dioxolan-4-yl)methoxy)carbonyl)oxy)- 3 -phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-131) and of the formula:

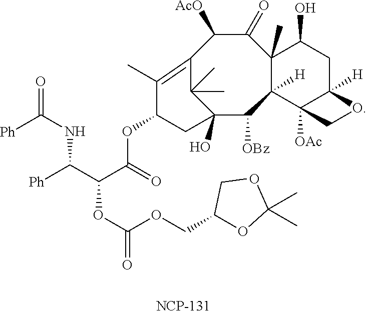

5. The acid labile lipophilic molecular conjugate of claim 1 that is (2aR,4S ,4aS ,6R,9S,11S,12S,12aR,12bS)-9-(((2R,3S)-3-benzamido-2-(((((R)-2,2-dimethyl-1,3 -dioxolan-4-yl)methoxy)carbonyl)oxy)- 3 -phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-132) and of the formula:

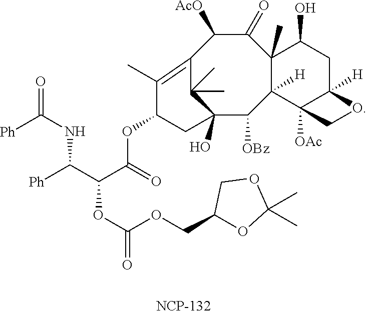

6. (canceled)

7. (canceled)

8. (canceled)

9. A pharmaceutical composition comprising: a) a therapeutically effective amount of a compound of claim 1, in the form of a single diastereoisomer; and b) a pharmaceutically acceptable excipient.

10. A method for the treatment of cancer in a patient comprising administering to the patient a therapeutically effective amount of a compound or composition of claim 1, to a patient in need of such treatment.

11. The method of claim 10, wherein the cancer is selected from the group consisting of leukemia, neuroblastoma, glioblastoma, cervical, colorectal, pancreatic, renal and melanoma.

12. The method of claim 10, wherein the cancer is selected from the group consisting of lung, breast, prostate, ovarian and head and neck.

13. The method of claim 10, wherein the method provides at least a 10% to 50% diminished degree of resistance expressed by the cancer cells when compared with the non-conjugated hydroxyl bearing cancer chemotherapeutic agent that is paclitaxel or cabazitaxel.

14. A method for reducing or substantially eliminating the side effects of chemotherapy associated with the administration of paclitaxel or cabazitaxel to a patient, the method comprising administering to the patient a therapeutically effective amount of an acid labile lipophilic molecular conjugate (ALLMC) of claim 1.

15. The method of claim 14, wherein the method provides a higher concentration of the paclitaxel or cabazitaxel in a cancer cell of the patient.

16. The method of claim 15, wherein the method delivers a higher concentration of paclitaxel or cabazitaxel in the cancer cell, when compared to the administration of a non-conjugated cancer chemotherapeutic agent that is paclitaxel or cabazitaxel to the patient, by at least 5%, 10%, 20% or at least 50%.

17. A stable, synthetic low density lipoprotein (LDL) solid nanoparticle comprising:
a) an acid labile lipophilic molecular conjugate (ALLMC) of the formulae: 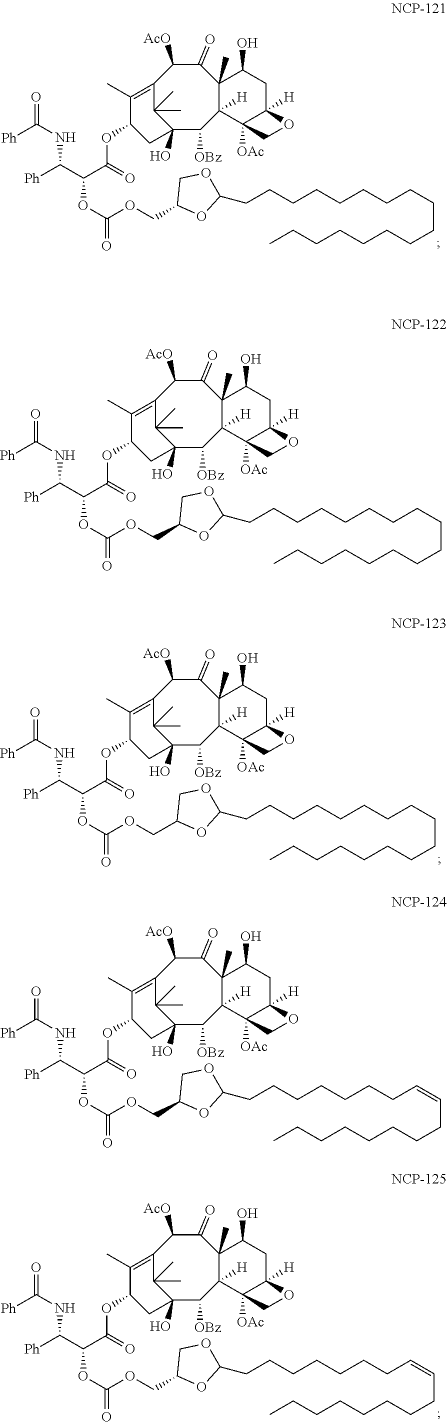 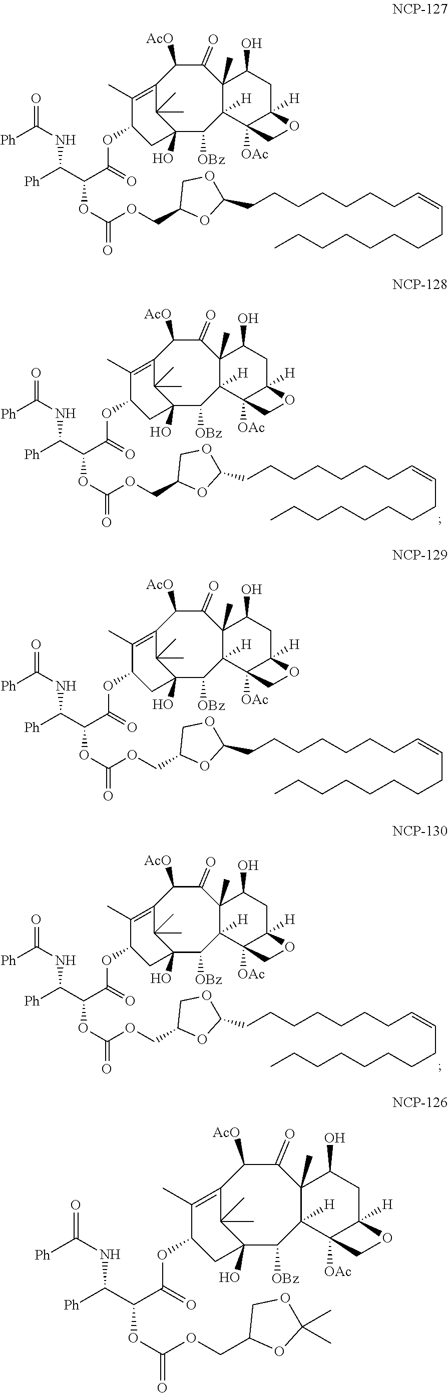 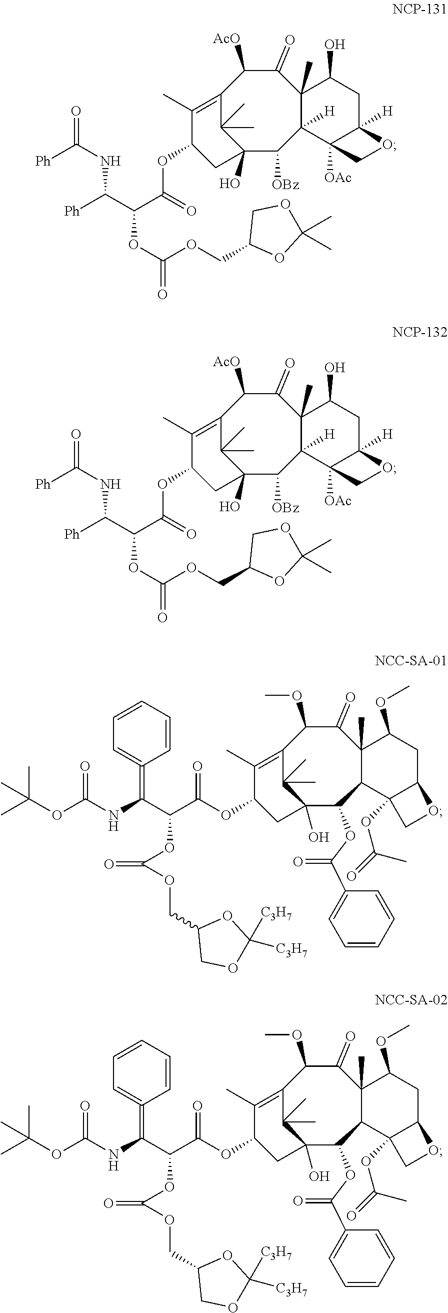 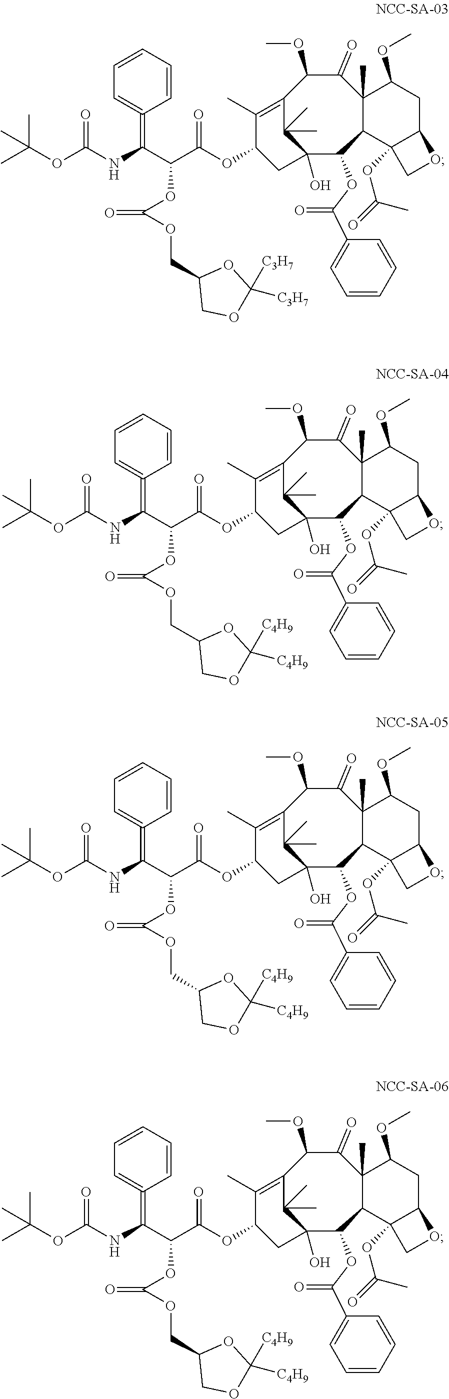 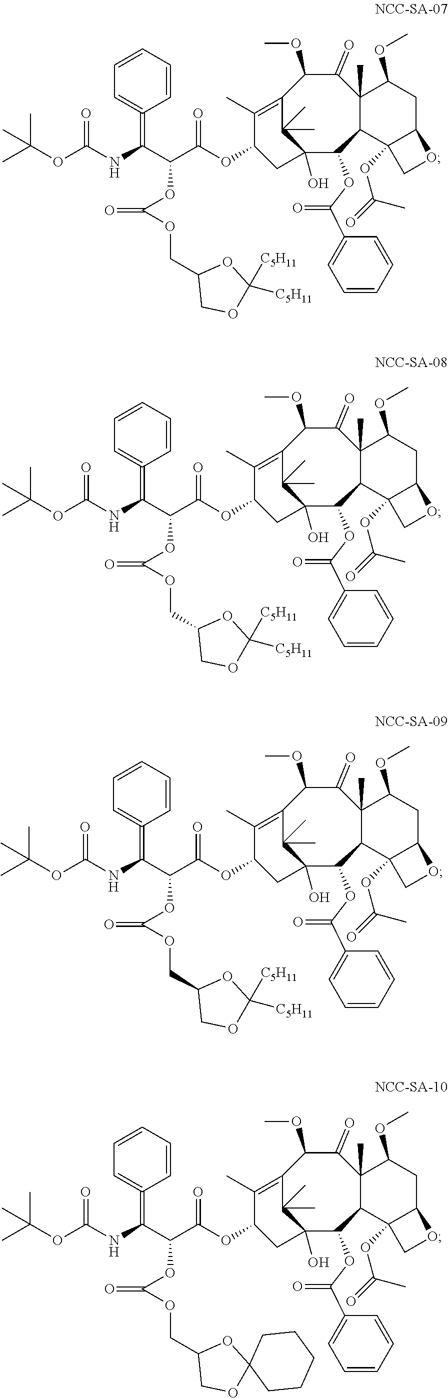 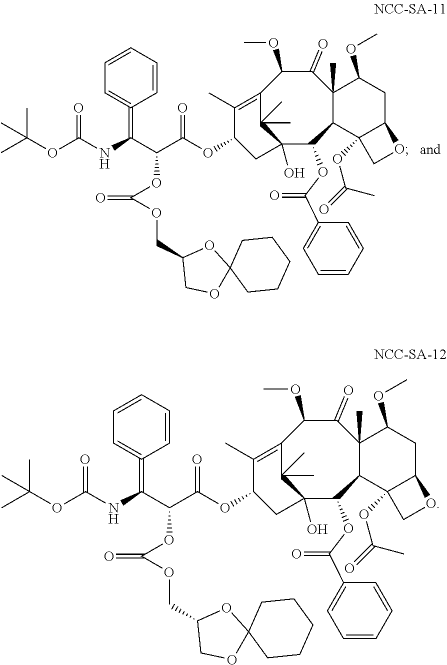
and their isolated diastereoisomers or mixtures thereof; or
a pharmaceutically acceptable salt thereof;
b) phospholipids (PL) wherein the phospholipids is selected from the group consisting of phosphotidylcholine, phosphotidylethanolamine, symmetric or asymmetric 1,2-diacyl-sn-glycero-3-phosphorylcholines, 1,2-dimyristoyl-sn-glycero-3-phosphorylcholine, 1,2-dimyristoyl-sn-glycero-3-phosphorylethanolamine, egg phospholipids, egg phosphatidyl glycerol, dipalmitoylphosphatidyl glycerol, egg lecithin, soy lecithin, lecithin (NOS) and mixtures thereof; and
c) a triglyceride (TG) selected from the group consisting of MIGLYOL 812 N, triacetin, tripropionin, tributyrin, triisovalerin, triisovalerin, tricapronin, triheptylin, tricaprylin, trinonylin, tricaprinin, and triundecylin;
wherein the LDL solid nanoparticle has a mean particle size of 40-80 nm.

18. The stable, synthetic low density lipoprotein (LDL) solid nanoparticle of claim 17, wherein the acid labile lipophilic molecular conjugate is (2aR,4S,4aS,6R,9S,11S,12S,12aR,12bS)-9-(((2R,3S)-3-benzamido-2-((((2,2-dimethyl-1,3-dioxolan-4-yl)methoxy)carbonyl)oxy)-3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2- b]oxete-6,12b(2aH)-diyl diacetate (NCP-126) and of the formula:

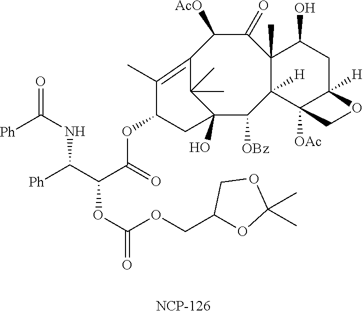

19. The stable, synthetic low density lipoprotein (LDL) solid nanoparticle of claim 17, wherein the acid labile lipophilic molecular conjugate is (2aR,4S ,4aS ,6R,9S,11S,12S,12aR,12bS)-9-(((2R,3S)-3-benzamido-2-(((((S)-2,2-dimethyl-1,3-dioxolan-4-yl)methoxy)carbonyl)oxy)- 3-phenylpropanoyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (NCP-131) and of the formula:

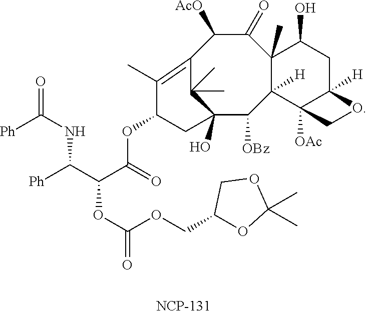

20. The stable, synthetic low density lipoprotein (LDL) solid nanoparticle of claim 17, wherein the acid labile lipophilic molecular conjugate is (2aR,4S ,4aS ,6R,9S,11S,12S,12aR,12bS)-9-(((2R,3S)-3-benzamido-2-(((((R)-2,2-dimethyl-1,3-dioxolan-4-yl)methoxy)carbonyl)oxy)- 3 -phenylpropanoyl)oxy)-12-(benzoyloxy)-4,11-dihydroxy-4a,8,13,13-tetramethyl-5-oxo-3 ,4,4a,5,6,9,10,11,12,12a-decahydro-1H-7,11-methanocyclodeca[3,4]benzo[1,2-b]oxete-6,12b(2aH)-diyl diacetate (NCP-132) and of the formula:

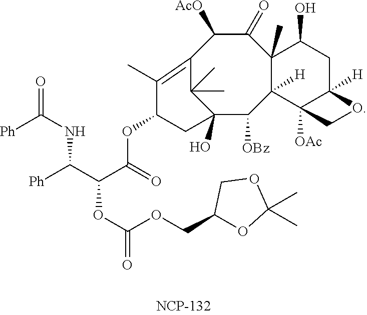

21. The stable, synthetic low density lipoprotein (LDL) solid nanoparticle of claim 17, wherein the nanoparticle has a mean size distribution of 60 nm.
